SEQUENCE LISTING (51 sequences)

SEQ ID NO: 1 - protein, 5 aa, Homo sapiens
KTTKS

SEQ ID NO: 2 - protein, 13 aa, Homo sapiens
YEALRVANEVTLN

SEQ ID NO: 3 - protein, 21 aa, Homo sapiens
HSNGLPLGGGSEEEAAAVVVK

SEQ ID NO: 4 - protein, 4 aa, Homo sapiens
RGDS

SEQ ID NO: 5 - protein, 5 aa, Homo sapiens
PHSRN

SEQ ID NO: 6 - protein, 4 aa, Homo sapiens
LDVP

SEQ ID NO: 7 - protein, 8 aa, Homo sapiens
WQPPRARI

SEQ ID NO: 8 - protein, 4 aa, Homo sapiens
REDV

SEQ ID NO: 9 - protein, 4 aa, Homo sapiens
IDAP

SEQ ID NO: 10 - protein, 15 aa, Homo sapiens
GTPGPQGIAGQRGVV

SEQ ID NO: 11 - protein, 6 aa, Homo sapiens
  misc_feature (3)..(3): Xaa is 4Hyp
GRXGER

SEQ ID NO: 12 - protein, 6 aa, Homo sapiens
GLKGEN

SEQ ID NO: 13 - protein, 6 aa, Homo sapiens
  misc_feature (3)..(3): Xaa is 4Hyp
GFXGER

SEQ ID NO: 14 - protein, 7 aa, Homo sapiens
MNYYSNS

SEQ ID NO: 15 - protein, 5 aa, Homo sapiens
EVTLN

SEQ ID NO: 16 - protein, 10 aa, Homo sapiens
ELDLSYNKLK

SEQ ID NO: 17 - protein, 5 aa, Homo sapiens
YIGSR

SEQ ID NO: 18 - protein, 5 aa, Homo sapiens
IKVAV

SEQ ID NO: 19 - protein, 12 aa, Homo sapiens
CCRRIKVAVWLC

SEQ ID NO: 20 - protein, 20 aa, Homo sapiens
HSNGLPLGGGSEEEAAAVVV

SEQ ID NO: 21 - protein, 21 aa, Homo sapiens
HSNGLPLGGGSEEEAAAVVVK

SEQ ID NO: 22 - DNA, 20 bp, Artificial Sequence
  Collagen 5 F primer
atcttccaaaggcccggatg

SEQ ID NO: 23 - DNA, 20 bp, Artificial Sequence
  Collagen 5 R primer
aaatgcagacgcagggtaca

SEQ ID NO: 24 - DNA, 19 bp, Artificial Sequence
  Vimentin F primer
cctcctaccgcaggatgtt

SEQ ID NO: 25 - DNA, 18 bp, Artificial Sequence
  Vimentin R primer
ctgtaggtgcgggtggac

SEQ ID NO: 26 - DNA, 20 bp, Artificial Sequence
  Decorin F primer
ctgcttgcacaagtttcctg

SEQ ID NO: 27 - DNA, 20 bp, Artificial Sequence
  Decorin R primer
gaccactcgaagatggcatt

SEQ ID NO: 28 - DNA, 20 bp, Artificial Sequence
  Keratocan F primer
tattcctggaaggcaaggtg

SEQ ID NO: 29 - DNA, 22 bp, Artificial Sequence
  Keratocan R primer
acctgcctcacacttctagacc

SEQ ID NO: 30 - DNA, 20 bp, Artificial Sequence
  Lumican F primer
cctggttgagctggatctgt

SEQ ID NO: 31 - DNA, 16 bp, Artificial Sequence
  Lumican R primer
taggatggccccagga

SEQ ID NO: 32 - DNA, 20 bp, Artificial Sequence
  ALDH3 F primer
ccccttcaacctcaccatcc

SEQ ID NO: 33 - DNA, 20 bp, Artificial Sequence
  ALDH3 R primer
gttctcactcagctccgagg

SEQ ID NO: 34 - DNA, 20 bp, Artificial Sequence
  MMP1 F primer
aggtctctgagggtcaagca

SEQ ID NO: 35 - DNA, 20 bp, Artificial Sequence
  MMP1 R primer
ctggttgaaaagcatgagca

SEQ ID NO: 36 - DNA, 20 bp, Artificial Sequence
  SMA F primer
ctgagcgtggctattccttc

SEQ ID NO: 37 - DNA, 20 bp, Artificial Sequence
  SMA R primer
ttctcaagggaggatgagga

SEQ ID NO: 38 - DNA, 19 bp, Artificial Sequence
  MMP2 F primer
ccaagcgtctagcaatacc

SEQ ID NO: 39 - DNA, 20 bp, Artificial Sequence
  MMP2 R primer
tctggggcagtccaaagaac

SEQ ID NO: 40 - DNA, 20 bp, Artificial Sequence
  MMP3 F primer
tgctttgtcctttgatgctg

SEQ ID NO: 41 - DNA, 20 bp, Artificial Sequence
  MMP3 R primer
aagcttcctgagggatttgc

SEQ ID NO: 42 - DNA, 21 bp, Artificial Sequence
  TIMP1 F primer
attgctggaaaactgcaggat

SEQ ID NO: 43 - DNA, 20 bp, Artificial Sequence
  TIMP1 R primer
tccacaagcaatgagtgcca

SEQ ID NO: 44 - DNA, 20 bp, Artificial Sequence
  RARA F primer
agtcctcaggctaccactat

SEQ ID NO: 45 - DNA, 20 bp, Artificial Sequence
  RARA R primer
cctccttcttcttcttgttt

SEQ ID NO: 46 - DNA, 20 bp, Artificial Sequence
  CD34 F primer
cttgggcatcactggctatt

SEQ ID NO: 47 - DNA, 20 bp, Artificial Sequence
  CD34 R primer
tccaccgttttccgtgtaat

SEQ ID NO: 48 - DNA, 20 bp, Artificial Sequence
  GAPDH F primer
agccgagccacatcgctgag

SEQ ID NO: 49 - DNA, 21 bp, Artificial Sequence
  GAPDH R primer
tgaccaggcgcccaatacgac

SEQ ID NO: 50 - DNA, 19 bp, Artificial Sequence
  Collagen 1 F primer
cctgcgtgtaccccactca

SEQ ID NO: 51 - DNA, 22 bp, Artificial Sequence
  Collagen 1 R Primer
accagacatgcctcttgtcctt

(19) United States
(12) Patent Application Publication
(10) Pub. No.: US 2023/0069065 A1
(43) Pub. Date: Mar. 2, 2023

(54) NOVEL CORNEAL TISSUES AND METHODS OF MAKING THE SAME

(71) Applicant: 3D Bio-tissues Limited, Newcastle Upon Tyne (GB)

(72) Inventors: Che John CONNON, Newcastle Upon Tyne (GB); Ricardo GOUVEIA, Newcastle Upon Tyne (GB)

(21) Appl. No.: 17/759,786

(22) PCT Filed: Jan. 29, 2021
(86) PCT No.: PCT/GB2021/050209

§ 371 Date: Jul. 29, 2022

(30) Foreign Application Priority Data
Jan. 30, 2020 (GB) 2001285.2

(51) Int. Cl. A61F 2/14; A61K 35/30; C07K 14/78; C12N 5/079

(52) U.S. Cl. CPC A61F 2/142; A61K 35/30; C07K 14/78; C12N 5/0621; A61K 38/00

(57) ABSTRACT

The invention relates to novel methods for making transparent and curved stromal cell tissues and decellularized forms thereof. Novel tissues are also provided.

### BACKGROUND

The creation of corneal stromal equivalents, particularly using natural components, typifies many of the challenges in tissue engineering. A suitable corneal stromal substitute needs to be biocompatible, mechanically stable and optically transparent, as well as capable of resisting contraction and proteolytic degradation before and after tissue transplantation (Ghezzi et al., 2015).

There is an increasing consensus that 3D geometries of the cell's environment are important regulators of cell behaviour (Li et al, 2014) and may thus be used as potential instructive cues in tissue regeneration. Specifically, substrate curvature has become recognised as an important factor in regulating cell and tissue phenotype during development (He and Jiang, 2017). In humans, at approximately week 8 of development, neural crest cells migrate across the lens and the cornea's presumptive structure, differentiating into both the corneal epithelium and stromal fibroblasts (Ligwale, 2015). The latter go on to synthesize the stroma's collagen and proteoglycan-rich extracellular matrix, as well as crystallins, a class of structural proteins essential for maintaining the tissue's transparency and refraction (Hassell and Birk, 2010). Variations in the viscoelastic properties and physical environment of the cornea during this period have been shown to play a vital role in the development of corneal curvature, which in turn affects the precise patterns of cell migration and differentiation involved in organogenesis (McMonnie and Boneham, 2007; Ligwale, 2015).

To date, some of the most promising corneal stromal substitutes are represented by collagen-based scaffold materials, which, once crosslinked, are shown to be usable as long-term corneal stromal replacements. Typically, these materials have been used in a top-down approach, with an initial step of hydrogel formation, followed by cell seeding, and matrix rearrangement after grafting (Fagerholm et al., 2014; Lewis et al., 2013; Rafat et al., 2008).

More recently, corneal stromal tissues have been engineered in vitro by means of bottom-up strategies, using the cells themselves to generate the extracellular matrix so as to recreate the well-defined, 3D microarchitecture of the native cornea (Wu et al., 2014, Gouveia et al., 2016). In this context, tissue templating presents several advantages over other methods, as it can direct cells to align, and in turn produce their own aligned collagen matrix, as well as promote easy tissue recovery with minimal manipulation. However, current bottom-up templating systems are still largely restricted to producing planar tissues which lack the natural curvature of the cornea and thus are not optimal for transplantation onto the eye.

Strategies to recreate geometrically curved stromal tissue equivalents are still rare and are generally based on (i) the production of collagen-based curved gels, to which cells are subsequently added (Li et al., 2003; Li et al., 2017) or (ii) applying mechanical strain to induce spherical curvature of planar collagen hydrogels containing corneal stromal cells (Zhang et al., 2017). However, these methods haven't resulted in improved tissue functionality, and relied on non-physiological conditions (i.e. animal-derived serum supplementation).

There is a clear need for improved methods of producing corneal stromal equivalents with improved function, particularly using natural components under physiological conditions.

### BRIEF SUMMARY OF THE DISCLOSURE

The inventors have previously demonstrated that curvature plays an important role in the highly-organised, anisotropic arrangement of stromal collagen, which in turn contributes to the mechanical and optical properties of the human cornea (Gouveia et al., 2017). Gouveia and colleagues showed that surface curvature induces human corneal stromal cells to migrate, align and subsequently deposit highly organised extracellular matrix (ECM) to form a single sheet of corneal-shaped stromal tissue equivalents.

The inventors have further investigated the ability of stromal cells to sense macroscopic changes in surface curvature. Suprisingly, the inventors have now found that, as well being able to sense and adapt to curvature of a surface with which they have direct contact (in 2D), stromal cells are also able to sense and adapt to curved surfaces that they do not directly contact (in 3D).

The invention is therefore based on the surprising finding that stromal cells can sense their macroscopic environment and adapt thereto even when the stromal cells are encapsulated or entrapped in a 3D hydrogel that is subsequently conformed to a curved surface. Suprisingly, the stromal cells are still able to sense the curvature of the surface and migrate, align and subsequently deposit highly organised extracellular matrix (ECM) within the 3D hydrogel structure. Furthemore, the cells can migrate, align and subsequently deposit highly organised ECM without the need for an alignment-inducing substrate (such as for example a silk fibroin film). Advantageously, the resultant 3D tissue maintains the transparent properties that were previously observed for single sheets of cells (Gouveia et al., 2017).

The tissues according to the present invention are thicker, denser and more organised whilst retaining the transparency observed for single sheets of curved stromal tissue. Such properties permit greater structural stability of the transparent and curved stromal cell tissues described herein. Advantageously, these tissues are more robust and therefore are easier to handle during transplantation procedures.

A method of making a transparent and curved stromal cell tissue having a minimum thickness of at least 50 μm is provided herein, the method comprising:

i) encapsulating or entrapping stromal cells in a hydrogel, wherein the hydrogel comprises cell adhesion motifs;

ii) transferring the hydrogel onto a curved surface; and

iii) retaining the hydrogel on the curved surface under appropriate cell culture conditions for at least twenty four hours to generate a curved stromal cell tissue with a minimum visible light transmittance value of at least 0.5, wherein the curved surface has a curvature of from about 0.04 to about 0.5 mm⁻¹.

Suitably, the cell adhesion motif may be an extracellular matrix protein sequence or a fragment or a variant thereof.

Suitably, the extracellular matrix protein may be selected from the group consisting of fibronectin, collagen, lumican, decorin, laminin, vitronectin, fibrinogen, elastin, biglycan, heparin, tenascin and osteopontin.

Suitably, the cell adhesion motif may be:

a) a fibronectin fragment comprising or consisting of an amino acid sequence selected from RGD (SEQ ID NO: 1), RGDS (SEQ ID NO: 5), PHSRN (SEQ ID NO: 6), LDVP (SEQ ID NO: 7), WQPPRARI (SEQ ID NO: 8), IGD (SEQ ID NO: 9), REDV (SEQ ID NO: 10), and IDAP (SEQ ID NO: 11) or a variant thereof;

b) a collagen fragment comprising or consisting of an amino acid sequence selected from KTTKS (SEQ ID NO:2), GTPGPQGIAGQRGVV (SEQ ID NO: 12), GROGER (SEQ ID NO: 13), GLKGEN (SEQ ID NO: 14), GFOGER (SEQ ID NO: 15), and MNYYSNS (SEQ ID NO: 16) or a variant thereof; or

c) a lumican fragment comprising or consisting of an amino acid sequence selected from EVTLN (SEQ ID NO: 17), ELDLSYNKLK (SEQ ID NO: 18) and YEALRVANEVTLN (SEQ ID NO: 3); or

d) a laminin fragment comprising or consisting of an amino acid sequence selected from the YIGSR (SEQ ID NO: 19), IKVAV (SEQ ID NO: 20), CCRRIKVAVWLC (SEQ ID NO: 21) and RGD.

Suitably, the hydrogel may have a total gel density of from about 0.25 to about 0.50 g/cm³.

Suitably, the hydrogel may have a gel hydration of from about 70% to about 90%.

Suitably, the hydrogel may have a gel stiffness of from about 0.5 to about 35×10⁶ Pa.

Suitably, the hydrogel may have a thickness of from about 0.1 to 12.0 mm.

Suitably, the hydrogel may have a collagen density of from about 0.07 to about 0.3 g/cm³.

Suitably, the hydrogel may be in the form of a thin layer or disc.

Suitably, the cell culture conditions may comprise retaining the hydrogel on the curved surface in cell culture medium.

Suitably, the cell culture medium may be Dulbecco's Modified Eagle Medium (DMEM), Ham's F12 or DMEM-F12.

Suitably, the cell culture medium may be serum free.

Suitably, the cell culture medium may comprise retinoic acid.

Suitably, the cell culture medium may comprise KTTKS lipopeptides in solution.

Suitably, the curved surface may be a polystyrene, polyethylene, polyethylene terephthalate, polylactic acid, polycarbonate, acrylonitrile butadiene styrene, agarose, a hydrogel or a glass surface. Optionally, the glass surface may be a borosilicate glass surface.

Suitably, the hydrogel may be maintained on the curved surface under appropriate cell culture conditions for at least four days.

Suitably, the stromal cells may be corneal stromal cells.

Suitably, the corneal stromal cells may be corneal fibroblasts.

Suitably, the stromal cells may be human.

Suitably, the stromal cells may be from a first species and the extracellular matrix protein sequence may be from a second species.

Suitably, the hydrogel comprising cell adhesion motifs may be generated using 3D bio-printing. Suitably, the stromal cell tissue may subsequently be decellularized.

A stromal cell tissue obtainable or obtained by a method described herein is also provided.

A decellularized tissue obtainable or obtained by a method described herein is also provided.

A stromal cell tissue having a minimum thickness of at least 50 μm, a minimum visible light transmittance value of at least 0.5 and a curvature of from about 0.04 to about 0.5 mm⁻¹ is also provided, wherein the tissue comprises:

a) stromal cells from a first species and

b) trace amounts of:
(i) an extracellular matrix protein sequence from a second species, or a fragment or a variant thereof; or
(ii) a synthetic extracellular matrix protein sequence or a fragment or a variant thereof.

Suitably, the first species may be human and the second species may be non-human.

Suitably, the stromal cells may be corneal stromal cells.

Suitably, the corneal stromal cells may be corneal fibroblasts.

A decellularized tissue having a minimum thickness of at least 50 μm, a minimum visible light transmittance value of at least 0.5 and a curvature of from about 0.04 and about 0.5 mm⁻¹ is also provided, wherein the tissue comprises:

a) extracellular matrix protein sequences from a first species and

b) trace amounts of:
(i) an extracellular matrix protein sequence from a second species, or a fragment or a variant thereof; or
(ii) a synthetic extracellular matrix protein sequence or a fragment or a variant thereof.

Suitably, the first species may be human, and the second species may be non-human.

Suitably, the tissue may have a thickness of from about 0.1 to 12.0 mm.

Suitably, the extracellular matrix protein sequence from a second species or synthetic extracellular matrix protein may be collagen.

A tissue is described herein for use in therapy. Optionally, the tissue is a composite tissue.

A tissue is described herein for use in keratoplasty. Optionally, the tissue is a composite tissue.

An in vitro model for studying a corneal disease or disorder comprising a tissue of the invention is also provided herein.

Use of a tissue of the invention as an in vitro corneal model is also provided. Suitably, the corneal model may be a corneal disease or disorder model.

A method of screening an agent for the treatment of a corneal disease or disorder is also provided comprising:
a) providing a tissue of the invention;
b) exposing said tissue to an agent; and
c) determining whether the agent has a therapeutic effect on the tissue.

A method of screening for any molecular or chemical agent is also provided, comprising:
a) providing a tissue of the invention;
b) exposing said tissue to an agent; and
c) determining whether the agent has a toxic effect on the tissue, or affects cell growth or viability, or alters gene expression in the tissue.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows a schematic representation of cell culture surfaces and methods used in this study. The different sizes of custom-made convex and concave surfaces (Ø=8, 12, 16, and 24 mm, corresponding to k=0.25, 0.16 (6), 0.125, and 0.083 (3) mm⁻¹, respectively, where the brackets refer to a recurring number) were used to determine the optimal and limiting range of meso-scale surface curvature able to affect corneal stromal cell behaviour. Normal glass coverslips were used as planar controls. The initial seeding of corneal stromal cells was delimited to a 500 μm-wide line along the surfaces' long (longitudinal) axis using Parafilm silicone wrap. This well-defined seeding point (inset) allowed for better monitoring cell migration patterns, for subsequent assignment (i.e., aligned if within 20° of either the long of short axis).

FIG. 2 shows bright-field and fluorescence microscopy of corneal stromal cells grown on different culture surfaces. Cells on planar and convex surfaces presented more stretched morphologies, whereas those on concave surfaces migrated faster, up to curvatures with Ø=16 mm (k=0.125 mm⁻¹). Both concave and convex curved surfaces promoted cell alignment. Scale bars, 100 μm.

FIG. 3 shows the migration rate of corneal stromal cells grown on different culture surfaces. Cells on planar and convex surfaces presented similar migration rates, independently of degree of curvature, whereas those on concave surfaces up to Ø=16 mm (k=0.125 mm⁻¹) migrated faster. Data correspond to average±s.d. of 3 experiments (n=3; n.s., not significant).

FIG. 4 shows corneal stromal cell alignment on different culture surfaces. Cells on convex and concave surfaces presented a high degree of cell alignment towards both the long (longitudinal) and small (arc) axis of curvature, up to Ø=16 mm (k =0.125 mm⁻¹) and optima at Ø=12 mm (k=0.16 (6) mm⁻¹). Cells grown on curved surfaces with Ø=24 mm (k=0.083(3) mm⁻¹) showed similar orientations to those on planar controls. Data correspond to average±s.d. of 3 experiments (n=3; n.s., not significant).

FIG. 5 shows nuclear circularity of corneal stromal cells grown on different culture surfaces. Cells on planar and convex surfaces presented similar lower nuclear circularity, independently of degree of curvature, whereas those on concave surfaces up to Ø=16 mm (k=0.125 mm⁻¹) showed significantly higher nuclear circularity compared to planar control. Data correspond to average±s.d. of 3 experiments (n=3; n.s., not significant).

FIG. 6 shows a summary of cellular effects promoted by planar, concave and convex culture surfaces.

FIG. 7 shows production and curvature induction on 3D collagen gels encapsulating corneal stromal cells. A) Schematic of the strategy used to realise and culture curved 3D collagen gels (top) and representative photographs (bottom panels) of such gels during the time in culture. B) Representative photographs of curved 3D collagen gels cultured for 7 days in serum-free medium. Scale bars: 500 μm.

FIG. 8 shows the effect of induced curvature on corneal stromal cell phenotype within 3D collagen gels. The mRNA of cells within curved or planar 3D collagen gels was extracted and analysed for the relative expression of gene markers of corneal A) extracellular matrix, B) fibroblast, C) myofibroblast, and D) protease/matrix turnover. Data (mean±SD) was obtained from three independent experiments (n=3), with statistical analysis performed using the Student's t-test (* corresponds to p<0.05).

FIG. 9 shows the effect of curvature on the optical properties of 3D collagen gels encapsulating corneal stromal cells. Graphs reporting the A) transmittance, B) reflectance, C) reduced scattering coefficient, and D) absorption coefficient show that curvature induced in culture (gray lines) increases collagen gel transparency compared to planar collagen gels (traced lines), to levels comparable to those of porcine corneas (black lines). Data (mean±SD) was obtained from two independent experiments (n=2), with statistical analysis performed using the Student's t-test (*, **, *** and **** corresponds to p<0.05, 0.01, 0.001 and 0.0001, respectively).

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

### Methods of Making a Tissue

The methods described herein are used to make a tissue. The tissues described herein may also be referred to as connective tissues. The terms “tissue” and “connective tissue” are therefore used interchangeably herein.

The term “connective tissue” encompasses connective tissues comprising stromal cells (i.e. “stromal cell connective tissues”) as well as connective tissues that have subsequently been decellularized (i.e. “decellularized connective tissue”). The terms “decellularized tissue” and “decellularized connective tissue” are therefore used interchangeably herein. Decellularized tissue may also be referred to as a “decellularized stromal cell tissue”, “tissue scaffold”, “connective tissue scaffold” or “extracellular matrix scaffold”.

A connective tissue is a continuous network or structure that binds tissues into their organ shape, supplies them with vessels and ducts and properly fastens the organs within the body cavity as well as binding organs to each other. Typically, a connective tissue comprises cells within an extracellular matrix (wherein the extracellular matrix further comprises fibrous proteins and non-fibrous ground substance). When a connective tissue is decellularized the cells are removed but the majority of the extracellular matrix components (i.e. the fibrous proteins and non-fibrous ground substance) are retained. Connective tissue comprising cells and decellularized forms thereof are readily identifiable by a person of skill in the art.

The ‘ground substance’ of an extracellular matrix is an amorphous gelatinous material. It is a clear, colourless, viscous fluid that fills the spaces between fibres and cells. It is composed of proteoglycans and cell adhesion proteins that allow the connective tissue to act as glue for the cells to attach to the matrix. The ground substance functions as a molecular sieve for substances to travel between blood capillaries and cells. Within the cornea it also affects the refractive index of the overall tissue.

The methods described herein may include making a tissue and subsequently decellularizing the tissue. The term “decellularization” refers to the process of removing the cells from a tissue whilst preserving the composition and structure of the extracellular matrix that has been produced by the cells. Accordingly, a decellularized tissue as described elsewhere herein refers to a tissue wherein the cells have been removed and wherein the composition and structure of the extracellular matrix that has been produced by the cells has been preserved. Suitable methods for decellularization are well known in the art. By way of non-limiting example, suitable methods of decellularization include physical, enzymatic, or chemical, or combinative methods.

The methods described herein use stromal cells to make a tissue. The term “stromal cell” refers to a connective tissue cell of an organ, for example a connective tissue cell of the eye, for example the connective tissue of the cornea, or a precursor of such a cell (for example a mesenchymal stromal cell). Stromal cells support the function of the parenchymal cells of the organ.

The methods described herein are particularly useful when used in combination with corneal stromal cells as they result in the production of a robust corneal stromal cell tissue that is optically transparent. The cornea is primarily composed of three cell types: epithelial cells, stromal cells, and endothelial cells, although other cell types may also be present (e.g., Langerhans and dendritic bone marrow-derived immune cells, trigeminal nerve dendrites, Schwann cells, and histiocytes).

Accordingly, in one example, the stromal cells are corneal stromal cells. Examples of suitable corneal stromal cells include corneal fibroblasts. Corneal fibroblasts, also known as keratocytes, are mesencyhmal-derived cells of the corneal stroma. The terms “corneal fibroblast” and “corneal keratocyte” are used interchangeably herein. The corneal stromal cells may also be corneal stem cells preferably comprising limbal epithelial cells, i.e. a heterogeneous mixture of stem cells and differentiated cells which is obtainable from the limbus at the edge of the cornea. In another example, the stromal cells include stromal progenitor cells such as corneal fibroblasts (keratocytes) in a differentiated or undifferentiated form. Preferably, these corneal fibroblasts are obtained from the peripheral limbus or from limbal rings. In another example, the stromal cells are bone marrow cells. The stromal cells may also be derived from iPSCs.

The corneal stroma is a dense connective tissue. It is the thickest layer of the cornea, and is located between the corneal epithelium and the inner endothelium of the cornea. It makes up the vast majority of the cornea and consists predominantly of 2 μm thick, flattened, collagenous lamellae (200-250 layers) oriented parallel to the corneal surface and continuous with the sclera at the limbus. Between the lamellae lie extremely flattened, modified fibroblasts known as keratocytes. Keratocytes (fibroblasts) secrete an extracellular matrix, which includes collagen and proteoglycans, and also produce crystalline proteins to maintain corneal transparency. These cells are stellate in shape with thin cytoplasmic extensions containing conspicuously few distinctive organelles when viewed in conventional cross-sections. However, frontal sections reveal an abundance of organelles and a novel network of fenestrations on their surface. Keratocytes are connected by gap junctions to their neighbouring cells and arranged in a corkscrew pattern spiralling from the epithelium to the endothelium.

The methods described herein are particularly useful for making a transparent and curved corneal stromal cell tissue using corneal stromal cells such as corneal fibroblasts.

In this context, a “transparent” tissue is a tissue with a minimum visible light transmittance value of at least 0.5. As used herein, visible light refers to light with a wave length of from about 380 nm to about 750 nm, in other words light that is visible to the human eye.

When visible light moves through a transparent (or semi-transparent) material, it can be transmitted, absorbed, or reflected. The term “visible light transmittance” as used herein, refers to the proportion of incident visible light that passes all the way through the tissue i.e. from the side where it entered to the side it emerged. For example, a tissue with a minimum visible light transmittance value of at least 0.5 is a tissue wherein at least 50% of the incident visible light is transmitted through the tissue to emerge at the other side. Typically, visible light transmittance is measured across the thickness of the tissue.

The transparent tissue as described herein is a tissue with a minimum visible light transmittance value of at least 0.5. In one example, the tissue has a minimum visible light transmittance value of at least 0.6. In another example, the tissue has a minimum visible light transmittance value of at least 0.7. In a further example, the tissue has a minimum visible light transmittance value of at least 0.8. In yet a further example, the tissue has a minimum visible light transmittance value of at least 0.9.

Methods for measuring visible light transmittance are well known in the art. For example, samples may be placed within a spectrophotometer between the incident light at a specified wavelength and the detector. The difference in intensity between the incident and detected light at specific wavelengths can be used to calculate transmittance values for the sample.

The stromal cells used in the methods described herein may be obtained directly from a living animal, methods for isolating stromal cells from animals are well known in the art.

The stromal cells may be human or from any other appropriate species (also referred to as a “first species” herein). The cells may be eukaryotic (e.g., animal, plant and fungal cells) or prokaryotic (e.g., bacterial cells). The cell may be an animal cell. For example, the cell is mammalian (for example human or mouse). Merely by way of example, a human cell may be a human stromal progenitor cell. In another example, the cells are fish cells. Non-limiting examples of suitable cell types include human cells, or cells from non-human primates, rodents (e.g. rats and mice), rabbits, horses, dogs, cats, sheep, cattle, pigs, fish or birds.

The stromal cells that are used in the methods described herein may comprise homogeneous or heterogeneous cell types. For example, the stromal cells may be a mixture of stromal cells from different species. For example, the stromal cells may include human stromal cells and additional stromal cells, for example, from one or more of non-human primates, rodents (e.g. rats and mice), rabbits, horses, dogs, cats, sheep, cattle, pigs, fish or birds. Alternatively, the stromal cells may be a mixture of stromal cells from any two or more of non-human primates, rodents (e.g. rats and mice), rabbits, horses, dogs, cats, sheep, cattle, pigs, fish or birds.

The methods provided herein are used to make a tissue comprising a curved surface having a curvature of from about 0.04 to about 0.5 mm⁻¹. For example, the methods provided herein are used to make a tissue comprising a curved surface having a curvature of from about 0.1 to about 0.4 mm⁻¹, for example, from about 0.2 to about 0.3 mm⁻¹. Standard methods for measuring curvature of a surface are well known in the art. For the avoidance of doubt, curvature as described herein is measured using the following methodology: k (curvature, in mm⁻¹)=1/R (radius of curvature, in mm). Advantageously, tissues comprising a curved surface having a curvature of from about 0.04 to about 0.5 mm⁻¹ (e.g. from about 0.2 to 0.3 mm⁻¹) mimic the natural curvature of the human corneal surface. Such tissues are therefore particularly useful for corneal therapies, which are discussed in more detail elsewhere herein.

The methods provided herein are used to make a tissue having a minimum thickness of at least about 50 μm. For example, the minimum thickness may be at least about 0.1 mm, at least about 0.5 mm or at least about 1 mm. As would be clear, the “thickness” of a tissue described herein is the distance between the outer tissue surface and the inner tissue surface, wherein the inner and outer tissue surfaces are both curved in the same orientation.

In one example, the tissue has a thickness of from about 50 μm to about 12.0 mm. For example, the thickness may be from about 0.1 to 12.0 mm, such as from about 0.1 to about 10 mm, from about 0.5 to about 5 mm, or from about 1.0 to about 2.0 mm. Each of these individual tissues (of thickness described) may be stacked to create composite tissues of resultant thickness from about 10 to about 50 mm.

The tissue may have any suitable volume. The tissue may, for example have a volume of from about 0.2 to about 100 ml, for example from about 0.2 to about 50 ml, such as from about 0.2 to about 25 ml or from about 0.2 to about 10 ml. In some examples, the volume of the tissue is from about 0.4 to about 5 ml, from about 0.4 to about 4 ml, or from about 0.4 to about 3 ml.

Individual stromal cell tissues generated by the methods described herein may be stacked to create a tissue composite. The term “stacked” as used herein, refers to layering two or more individual tissues. For example, a stromal cell tissue generated by the methods described herein may be stacked upon a second stromal cell tissue generated by the methods described to form a tissue composite. Appropriate methods for stacking tissues are well known in the art.

In one example, 1,000 or more individual stromal cell tissues generated by the methods described herein may be stacked to create a tissue composite.

In another example, 2,000 or more individual stromal cell tissues may be stacked to create a tissue composite. For example, 3,000 or more, or 4000 or more individual stromal cell tissues may be stacked to create a tissue composite. In a further example, 5,000 or more individual stromal cell tissues generated by the methods described herein may be stacked to create a tissue composite.

As would be clear to a person of skill in the art, any suitable number of individual stromal cell tissues may be stacked to create a tissue composite of a particular thickness. Suitable tissue composite thicknesses are described elsewhere herein.

As used herein, “tissue composite” refers to a tissue that is generated from two or more elements wherein each element comprises an individual tissue and/or cell layer. For example, as described elsewhere herein, a tissue composite may be created by stacking two or more (e.g. 1,000 or more, 2,000 or more, 3,000 or more, 4,000 or more, 5,000 or more) individual stromal cell tissues generated by the methods provided herein. In an alternate example, a tissue composite may be created from an endothelial or epithelial cell layer and an individual stromal cell tissue.

Tissue composites generated by the methods described herein may be of any suitable thickness. For example, tissue composites may differ in thickness depending on their intended use (i.e. a tissue composite for use in keratoplasty may have a different thickness from a tissue composite for use in cardiac surgery). A suitable thickness for a tissue composite would be readily identifiable by a person of skill in the art.

In a non-limiting example, the tissue composite generated by the methods described herein has a thickness of from about 10 mm to about 100 mm.

In a particular example, the tissue composite generated by the methods described herein has a thickness of from about 10 mm to about 50 mm.

In another example, the tissue composite generated by the methods described herein may have a thickness of from about 15 mm to about 45 mm. In a further example, the tissue composite may have a thickness of from about 20 mm to about 40 mm, or from about 25 mm to about 35 mm.

The methods described herein comprise the step of encapsulating or entrapping stromal cells in a hydrogel. The term “hydrogel” as used herein refers a structure made from cross-linked polymers. The hydrogel may be insoluble in water but may be capable of absorbing and retaining large quantities of water to form a stable, often soft and pliable, structure. The hydrogel may comprise internal pores.

In one example, the hydrogel is a reversibly cross-linked hydrogel. As used herein a “reversibly cross-linked hydrogel” refers to a hydrogel that is formed by reversible cross-linking (i.e. the cross-linking can be reversed such that the hydrogel reverts back to a solution). Reversal of the cross-linking enables the entrapped or encapsulated cells/tissue to be released from the hydrogel (e.g. at their point of use). Examples of reversibly cross-linked hydrogels are well known in the art. Accordingly, suitable hydrogels may readily be identified by a person of skill in the art.

The hydrogel referred to herein may comprise a hydrogel-forming polymer having a cross-linked or network structure or matrix; and an interstitial liquid. The term “hydrogel-forming polymer” refers to a polymer which is capable of forming a cross-linked or network structure or matrix under appropriate conditions, wherein an interstitial liquid and the entrapped or encapsulated stromal cells may be retained within such a structure or matrix. Initiation of the formation of the cross-linked or network structure or matrix may be by any suitable means, depending on the nature of the polymer.

The polymer will in general be a hydrophilic polymer. It will be capable of swelling in an aqueous liquid. In one example, the hydrogel-forming polymer is collagen.

As used herein, the term “collagen” refers to the main protein of connective tissue that has a high tensile strength, and is found in most multicellular organisms. Collagen is a major fibrous protein, and it is also the nonfibrillar protein in basement membranes. It contains an abundance of glycine, proline, hydroxyproline, and hydroxylysine. In the context of the present disclosure, collagen includes any one or more types of collagen, whether native nor not, for example atelocollagen, insoluble collagen, collagen fibres, soluble collagen, and acid-soluble collagen. There are currently at least 28 types of collagen identified which are all encompassed herein. Collagen may be for example fibrillar or non-fibrillar. Fibrillar collagen may be, for example, type I, II, III, V, XI. Non-fibrillar collagen may be, for example, fibril associated collagen with interrupted triple helices (type IX, XII, XIV, XIX, XXI), short chain collagen (type VIII, X), basement membrane collagen (Type IV), multiplexin (XV, XVIII), membrane associated collagen with interrupted triple helices (type XIII, XVII), and collagen type VI and type VII. In one example, the collagen may be type I collagen or type III collagen.

Typically, a collagen hydrogel comprises a matrix of collagen fibrils which form a continuous scaffold around an interstitial liquid and the entrapped or encapsulated stromal cells/tissue. Dissolved collagen may be induced to polymerise/aggregate by the addition of dilute alkali to form a gelled network of cross-linked collagen fibrils. The gelled network of fibrils supports the original volume of the dissolved collagen fibres, retaining the interstitial liquid. General methods for the production of such collagen gels are well known in the art (e.g. WO2006/003442, WO2007/060459 and WO2009/004351).

Typically, collagen which is used in a collagen hydrogel may be any fibril-forming collagen. Examples of fibril-forming collagens are Types I, II, III, V, VI, IX and XI. The hydrogel may comprise all one type of collagen or a mixture of different types of collagen. Preferably, the hydrogel comprises or consists of Type I collagen or Type V collagen (which are responsible for maintaining the transparency of the cornea). In some examples, the hydrogel is formed exclusively or substantially from collagen fibrils, i.e. collagen fibrils are the only or substantially the only polymers in the hydrogel. In other examples, the collagen hydrogel may additionally comprise other naturally-occurring polymers, e.g. silk, fibronectin, elastin, chitin and/or cellulose. Generally, the amounts of the non-collagen naturally-occurring polymers will be less than 5%, preferably less than 4%, 3%, 2% or 1% of the hydrogel (wt/wt). Similar amounts of non-natural polymers may also be present in the hydrogel, e.g. peptide amphiphiles, polylactone, polylactide, polyglycone, polycaprolactone and/or phosphate glass.

In some examples, the hydrogel-forming polymer is alginic acid or an alginate salt of a metal ion. Preferably, the metal is a Group 1 metal (e.g. lithium, sodium, or potassium alginate) or a Group 2 metal (e.g. calcium, magnesium, barium or strontium alginate). Preferably, the polymer is calcium alginate or sodium alginate or strontium alginate, most preferably calcium alginate.

In some examples, the hydrogel-forming polymer is an alginate. In other examples, the hydrogel-forming polymer is a mixture of alginate and another hydrogel-forming polymer. In some examples, the alginate is modified (e.g. with peptides).

In yet other examples, the hydrogel-forming polymer is a cross-linked acrylic acid-based (e.g. polyacrylamide) polymer.

In yet further examples, the hydrogel-forming polymer is a cross-linkable cellulose derivative, a hydroxyl ether polymer (e.g. a poloxamer), pectin or a natural gum.

In some examples, the hydrogel is not thermo-reversible at physiological temperatures, i.e. the sol-gel transition of the hydrogel cannot be obtained at a temperature of 0-40° C.

The structure of the hydrogel may be changed by varying the concentration of the hydrogel-forming polymer in the hydrogel. The structure affects the viability of the cells in the hydrogel, the rate of cellular differentiation as well as the robustness of the gel and its handling properties. Preferred concentrations of the hydrogel-forming polymer in the hydrogel are 0.2-5% (weight of polymer to volume of interstitial liquid), and include for example 0.2-0.4%, 0.4-0.5%, 0.5-0.7%, 0.7-1.1%, 1.1-1.3%, 1.3-2.2%, 2.2-2.6%, 2.6-3.0%, 3.0-3.5%, 3.5-4.0%, 4.0-4.5% and 4.5-5.0% (or any combination thereof e.g. 0.2-0.5%, 0.2 to 0.7% etc).

In one example, the viscosity of the non-gelled hydrogel solution is up to 500 mPa·s, Optionally, the viscosity of the non-gelled hydrogel solution is between 5 and 200 mPa·s (preferably between 5 and 100 mPa·s).

In other examples, the concentration of the hydrogel-forming polymer in the hydrogel is above 0.25%, 0.3%, 0.4%, 0.5% or 0.6%. In other examples, the concentration of the hydrogel-forming polymer in the hydrogel is below 5%, 4.5%, 4.0%, 3.5%, 3.0%, 2.6%, 2.4%, 1.5%, 1.4%, 1.3% or 1.2%. In some preferred examples, the concentration of the hydrogel-forming polymer in the hydrogel is about 0.3%, about 0.6% or about 1.2%. In some particular examples, the concentration of the hydrogel-forming polymer in the hydrogel is about 1%. In some particular examples, the hydrogel is formed from about 1% sodium alginate or from about 1% calcium alginate, or from about 1.2% strontium alginate.

In some examples, the gelling of the hydrogel is facilitated using a compound comprising a multivalent metal cation, e.g. using calcium chloride. In particular, calcium chloride (e.g. 50-200 mM calcium chloride, preferably 75-120 mM calcium chloride) may be used to gel alginate hydrogels.

In other examples, an alternative metal chloride is used, e.g. magnesium or barium or strontium chloride. Alternatively, other multivalent cations may be used, e.g. La³⁺ or Fe³⁺

In some examples, the gels (preferably alginate gels) additionally comprise CO₂.

In some examples, the hydrogel comprises cross-linked alginate. For example, the hydrogel may comprise cross-linked calcium-alginate, strontium-alginate, barium-alginate, magnesium-alginate or sodium-alginate. In one particular example, the cross-linked alginate is from about 0.5% (w/v) to about 5.0% (w/v) calcium alginate. For example, the cross-linked alginate may be from about 1.0% (w/v) to about 2.5% (w/v), about 1.5% (w/v) to about 2.0% (w/v) calcium alginate, or any range therebetween.

The interstitial liquid may be any liquid in which polymer may be dissolved and in which the polymer may gel. Generally, it will be an aqueous liquid, for example an aqueous buffer or cell culture medium. The liquid may contain an antibiotic. Preferably, the hydrogel is sterile, i.e. aseptic. Preferably, the liquid does not contain animal-derived products, e.g. foetal calf serum or bovine serum albumin.

It has been found that the mechanical strength of the hydrogel may be enhanced by the encapsulation of a reinforcing structure, scaffold or mesh within the gel. It may be synthetic or natural polymer. Preferably, the reinforcing structure, scaffold or mesh is biodegradable. The reinforcing structure, scaffold or mesh may, for example be a polymer comprising polylactic acid (e.g. poly-(lactic acid-co-caprolactone) (PLACL)), collagen or nylon.

Hydrogels that are used in the methods described herein are typically in the form of a thin layer or disc. Wth regard to a hydrogel disc, the preferred hydrogel polymer concentration is about 1.2% due to the increased structural stability provided by this concentration. Preferably, the hydrogel (e.g. a disc) is an uncompressed hydrogel, i.e. it has not been subjected to an axial compressing force.

In some examples, the hydrogel may be extruded via a needle to form a thin layer or disc. In other words, the hydrogel may be generated via 3D printing. Standard methods for 3D printing are well known in the art.

Hydrogels that are used in the methods described herein typically have a total gel density of from about 0.25 to about 0.50 g/cm³. For example, a total gel density of from about 0.35 to about 0.48 g/cm³ may be considered, or a total gel density of from about 0.38 to about 0.45 g/cm³ may be used. An optimum value within this range may also be chosen e.g. about 0.4 g/cm³.

Hydrogels that are used in the methods described herein typically have a gel hydration of from about 70% to about 90%. For example, a gel hydration of from about 73% to about 86% may be considered, or a gel hydration of from about 75% to about 80% may be used. An optimum value within this range may also be chosen e.g. about 76%.

Hydrogels that are used in the methods described herein typically have a gel stiffness of from about 0.5 to about 35×10⁶ Pa. For example, a gel stiffness of from about 0.5 to about 20×10⁶ Pa may be considered, or a gel stiffness of from about 0.5 to about 10×10⁶ Pa may be used. For example, a gel stiffness of from about 0.5 to about 5.5×10⁶ Pa may be considered, or a gel stiffness of from about 2 to about 5×10⁶ Pa may be used. An optimum value within this range may also be chosen e.g. about 4×10⁶ Pa.

Hydrogels that are used in the methods described herein typically have a thickness of from about 0.1 to 12.0 mm. For example, the hydrogel thickness may be from about 0.1 to about 10 mm, from about 0.5 to about 5 mm, or from about 1.0 to about 2.0 mm. The hydrogel may have any suitable volume. The hydrogel may, for example have a volume of from about 0.2 to about 100 ml, for example from about 0.2 to about 50 ml, such as from about 0.2 to about 25 ml or from about 0.2 to about 10 ml. In some examples, the volume of the hydrogel is from about 0.4 to about 5 ml, from about 0.4 to about 4 ml, or from about 0.4 to about 3 ml.

As detailed elsewhere herein, the hydrogels that are used in the methods described herein may comprise collagen. Collagen hydrogels typically have a collagen density of from about 0.07 to about 0.3 g/cm³. For example, a collagen density of from about 0.07 to about 0.2 g/cm³ may be considered, or a collagen density of from about 0.08 to about 0.15 g/cm³ may be used. An optimum value within this range may also be chosen e.g. about 0.1 g/cm³.

In the context of collagen hydrogels specifically, a total gel density of from about 0.35 to about 0.48 g/cm³ may be considered (e.g. from about 0.38 to about 0.45 g/cm³ preferably about 0.4 g/cm³). In addition, a gel hydration of from about 73% to about 86% may be considered, (e.g. from about 75% to about 80%, preferably about 76%). Furthermore, a gel stiffness of from about 0.5 to about 5.5×10⁶ Pa may be considered, (e.g. from about 2 to about 5×10⁶ Pa, preferably about 4×10⁶ Pa).

The methods described herein comprise encapsulating or entrapping stromal cells (e.g. corneal stromal cells such as keratocytes) in a hydrogel. As used herein, the term “entrapped” refers to the stromal cells being physically captured/trapped by the hydrogel, such that they are not released from the hydrogel (unless for example the cross-linking in the hydrogel is reversed such that the hydrogel reverts to a solution). The stromal cells may be entrapped by virtue of being completely surrounded by the hydrogel, or they may be entrapped by virtue of the majority (but not all) of the cell being surrounded by the hydrogel. In this context, the “majority” refers to at least about 50%, at least about 60%, at least about 70%, at least about 80%, at least about 85%, at least about 90%, at least about 95%, at least about 96%, at least about 97%, at least about 98%, or at least about 99% of the cell (by volume) being surrounded by the hydrogel. In this context, “completely surrounded” refers to about 100% of the cell (by volume) being surrounded by the hydrogel. The term “encapsulated” refers to enclosing the stromal cells in the hydrogel. A cell is “encapsulated” by a hydrogel when it is completely surrounded by the hydrogel. An “encapsulated” cell is also an “entrapped” cell. By contrast, an “entrapped” cell may or may not be “encapsulated” by the hydrogel.

The hydrogel may be formed separately from the stromal cells, such that the cells are added to an already gelled hydrogel (i.e. a hydrogel wherein crosslinking has already occurred). Alternatively, the hydrogel may be formed in the presence of the stromal cells. Methods for making hydrogels and entrapping or encapsulating cells within such hydrogels are well known in the art.

For example, the hydrogel may be formed by polymerising the hydrogel-forming polymer in the presence of the stromal cells to form a hydrogel wherein the stromal cells are entrapped or encapsulated within the hydrogel itself. Methods for polymerising the hydrogel-forming polymer to form a hydrogel are well known in the art, and differ depending on the polymer used. For example, polymerisation of an alginate solution (to form an alginate hydrogel) may be induced by a chemical agent such as calcium chloride. As used herein, the terms “polymerising” and “gelling” the hydrogel are used interchangeably to refer to the change in state of the hydrogel-forming polymer from a liquid to a hydrogel. The hydrogel may be gelled under appropriate cell-compatible conditions, i.e. conditions which are not detrimental or not significantly detrimental to the viability of the cells. In some examples, the hydrogels are prepared under cGMP (current Good Manufacturing Practice) conditions.

The stromal cells may be seeded into the hydrogel during the formation of the hydrogel from its constituent polymers, for example, by mixing the cells with a solution of the monomer prior to polymerization/aggregation or prior to cross-linking of a hydrogel-forming polymer. For this reason, the hydrogel may be gelled under appropriate cell-compatible conditions, i.e. conditions which are not detrimental or not significantly detrimental to the viability of the cells. Accordingly, the methods described herein may include the step of forming a hydrogel in the presence of the stromal cells such that the stromal cells are encapsulated or entrapped within the hydrogel. The invention further provides a process for preparing a hydrogel, comprising the step of gelling the hydrogel-forming polymer (for example using a Group 2 metal salt selected from the group consisting of magnesium and calcium salts).

In some examples, the concentration of cells which are present in the hydrogel is 1×10³-1×10⁶ cells/ml hydrogel solution. Generally the concentration of cells is less than 5×10⁵, preferably 0.1×10⁵-5×10⁵ cells/ml hydrogel, more preferably 0.5×10⁵-2.3×10⁵ cells/ml hydrogel, and most preferably 1.0×10⁵-2.0×10⁵ cells/ml hydrogel.

In one example, a reversibly cross-linked hydrogel may be used. A reversibly cross-linked hydrogel is one from which living cells can be released. Accordingly, the method may include the step of dissociating the hydrogel to release the stromal cell tissue after it has been made. In other words, after the stromal cell tissue has been made, the hydrogel is capable of being dissociated thus allowing the release or removal of all or substantially all of the cells which were previously retained therein. The hydrogel is dissociated under appropriate cell-compatible conditions, i.e. conditions which are not detrimental or not significantly detrimental to the cells. Preferably, the hydrogel is dissociated by being chemically disintegrated or dissolved.

The hydrogels of the methods described herein comprise cell adhesion motifs. As used herein, the term “cell adhesion motif” encompasses extracellular matrix protein motifs, and fragments and variants thereof, which are involved in cell adhesion. The term “cell adhesion motif” therefore encompasses extracellular matrix protein sequences, or fragments or variants thereof, where the fragments or variants are involved in cell adhesion. As used herein, “involved in cell adhesion” refers to promoting cell adhesion, and/or directly adhering or binding to the cell e.g. by binding to cells via cell surface molecules, such as integrins, displayed on the surface of the cells. Cell adhesion motifs are typically capable of adhering to cells directly, for example, by binding to cells via cell surface molecules, such as integrins, displayed on the surface of the cells.

In one example, the cell adhesion motif is an extracellular matrix protein sequence or a fragment or a variant thereof.

Many extracellular matrix proteins involved in cell adhesion are known in the art. By way of example, an extracellular matrix protein involved in cell adhesion may be selected from the group consisting of fibronectin, collagen (such as types I, II, III and V), lumican, decorin, laminin, vitronectin, gelatin, chitosan, fibrin, fibrinogen, elastin, biglycan, heparin, tenascin and osteopontin. Additionally, the cell adhesion motifs may be any peptide derived from any of the aforementioned proteins, including derivatives or fragments containing the binding domains of the above-described molecules. Example motifs include integrin-binding motifs, such as the RGD (SEQ ID NO:1—arginine-glycine-aspartate) motif, the YIGSR (SEQ ID NO: 19—tyrosine-isoleucine-glycine-serine-arginine) motif, and related peptides that are functional equivalents. For example, peptides containing RGD (SEQ ID NO:1) sequences (e.g., RGDS (SEQ ID NO:5)) and WQPPRARI (SEQ ID NO:8) sequences are known to direct spreading and migration properties of endothelial cells, and YIGSR (SEQ ID NO:19) peptide has been shown to promote epithelial cell attachment. Whether an amino acid sequence is a cell adhesion motif can be determined by screening peptide libraries for adhesion and selectivity to specific cell types. Cell adhesion motifs may also be developed empirically via Phage display technologies.

The hydrogel may comprise an extracellular matrix protein sequence (i.e. a sequence of an extracellular matrix protein; also referred to as a motif) that is involved in cell adhesion. The hydrogel may alternatively comprise fragments or variants of such sequences, where the fragments or variants are also involved in cell adhesion. Such sequences (including fragments and variants thereof) are referred to herein as “cell adhesion motifs”.

The hydrogel may comprise one or more different cell adhesion motifs. For example, the hydrogel may comprise 2, 3, 4, 5 or more different cell adhesion motifs. In an example where the hydrogel comprises more than one cell adhesion motif.

The cell adhesion motif may be an extracellular matrix protein or a fragment or variant thereof that is involved in cell adhesion (i.e. an extracellular matrix protein that is involved in cell adhesion; a variant of the extracellular matrix protein, wherein the variant is involved in cell adhesion; a fragment of the extracellular matrix protein, wherein the fragment is involved in cell adhesion; or a variant of a fragment of the extracellular matrix protein, wherein the variant of the fragment is involved in cell adhesion).

The cell adhesion motif may be a fibronectin fragment that comprises or consists of an amino acid sequence selected from the group consisting of RGD (SEQ ID NO:1), RGDS (SEQ ID NO:5), PHSRN (SEQ ID NO:6), LDVP (SEQ ID NO:7), WQPPRARI (SEQ ID NO:8), IGD (SEQ ID NO:9), REDV (SEQ ID NO: 10), and IDAP (SEQ ID NO:11) or a variant thereof. The variant may be a conservative amino acid substitution variant e.g. having one, two or three conservative amino acid substitutions compared to an amino acid sequence selected from the group consisting of RGD (SEQ ID NO:1), RGDS (SEQ ID NO:5), PHSRN (SEQ ID NO:6), LDVP (SEQ ID NO:7), WQPPRARI (SEQ ID NO:8), IGD (SEQ ID NO:9), REDV (SEQ ID NO: 10), and IDAP (SEQ ID NO:11).

Alternatively, the cell adhesion motif may be a collagen fragment that comprises or consists of an amino acid sequence selected from the group consisting of KTTKS (SEQ ID NO:2), GTPGPQGIAGQRGVV (SEQ ID NO:12), GROGER (SEQ ID NO:13), GLKGEN (SEQ ID NO:14), GFOGER (SEQ ID NO:15), and MNYYSNS (SEQ ID NO:16) or a variant thereof. The variant may be a conservative amino acid substitution variant e.g. having one, two or three conservative amino acid substitutions compared to an amino acid sequence selected from the group consisting of KTTKS (SEQ ID NO:2), GTPGPQGIAGQRGVV (SEQ ID NO:12), GROGER (SEQ ID NO:13), GLKGEN (SEQ ID NO:14), GFOGER (SEQ ID NO:15), and MNYYSNS (SEQ ID NO:16).

Alternatively, the cell adhesion motif may be a lumican fragment that comprises or consists of an amino acid sequence selected from the group consisting of EVTLN (SEQ ID NO:17), ELDLSYNKLK (SEQ ID NO:18) and YEALRVANEVTLN (SEQ ID NO:3) or a variant thereof. The variant may be a conservative amino acid substitution variant e.g. having one, two or three conservative amino acid substitutions compared to an amino acid sequence selected from the group consisting of EVTLN (SEQ ID NO:17), ELDLSYNKLK (SEQ ID NO:18) and YEALRVANEVTLN (SEQ ID NO:3).

Alternatively, the cell adhesion motif may be a laminin fragment that comprises or consists of an amino acid sequence selected from the group consisting of YIGSR (SEQ ID NO:19), IKVAV (SEQ ID NO:20), CCRRIKVAVWLC (SEQ ID NO:21) and RGD (SEQ ID NO:1) or a variant thereof. The variant may be a conservative amino acid substitution variant e.g. having one, two or three conservative amino acid substitutions compared to an amino acid sequence selected from the group consisting of YIGSR (SEQ ID NO:19), IKVAV (SEQ ID NO:20), CCRRIKVAVWLC (SEQ ID NO:21) and RGD (SEQ ID NO:1).

The cell adhesion motifs may be natural or synthetic. A synthetic amino acid sequence may resemble an amino acid sequence, for example a peptide, that occurs in nature. Merely by way of example, a synthetic cell adhesion motif may be selected from the group consisting of V₂A₂E₂, HSNGLPLGGGSEEEAAAVW (SEQ ID NO: 22), HSNGLPLGGGSEEEAAAWV(K) (SEQ ID NO: 23) and HSNGLPLGGGSEEEAAAVWK (SEQ ID NO: 4) or a variant thereof.

The fragment or variant of a cell adhesion protein may substantially retain the biological function of the corresponding wild type peptide. The term “biological function” as used herein may refer to the ability to promote cell binding. By “substantially retains” biological function, it is meant that the fragment or variant retains at least about 50%, 60%, 75%, 85%, 90%, 95%, 97%, 98%, 99%, or more, of the biological function of the wild type peptide to, for example, promote cell binding. Indeed, the fragment or variant may have a higher biological function than the wild type peptide. The fragment or variant may have 110%, 120%, 130%, 140%, 150%, 160%, 170%, 180%, 190%, 200%, or more, of the biological function of the wild type peptide to, for example, promote cell binding.

The extracellular matrix protein (or a fragment or variant thereof) described herein may be from any appropriate species. In some examples, the extracellular matrix protein (or a fragment or variant thereof) that is present within the hydrogel is from a different species (also referred to as a “second species”) to the stromal cells that are entrapped or encapsulated in the hydrogel (where the stromal cells are from a first species). In other words, the species of the stromal cells in the hydrogel may be distinct to that of the cell adhesion motifs (or corresponding extracellular matrix protein) present within the hydrogel. This results in a tissue comprising stromal cells from a first species and trace amounts of extracellular matrix protein sequence (or a fragment or variant thereof) from a second (distinct) species.

In other words, the tissue described herein may comprise stromal cells from a first species and trace amounts of extracellular matrix protein sequence (or a fragment or variant thereof) from a second species, wherein the first species and second species are not the same. Examples of such tissues include tissues with human stromal cells and trace amounts of porcine extracellular matrix protein sequence (or a fragment or variant thereof), or vice versa (i.e. tissues with porcine stromal cells and trace amounts of human extracellular matrix protein sequence (or a fragment or variant thereof)). Other suitable combinations of cells and extracellular matrix protein sequence (or a fragment or variant thereof) may readily be identified by a person of skill in the art.

The methods provided herein typically result in a tissue that comprises trace amount of the extracellular matrix protein sequence that was originally part of the hydrogel used to entrap or encapsulate the stromal cells. As used herein, “trace amounts” of the extracellular matrix protein sequence refers to detectable levels of the extracellular matrix protein sequence, using standard methods such as immunohistochemistry, immunoblotting/ELISA, mass spectroscopy, zymography, etc.

As described elsewhere herein, the stromal cell tissues that are made by the methods of the invention may also be decellularized. Similar to the stromal cell tissues described above, decellularized tissues may also comprise components from at least two different species. For example, when the stromal cell tissue (before decellularization) is generated using a hydrogel with extracellular matrix protein sequence (or a fragment or variant thereof) from a different species (also referred to as a “second species”) to the stromal cells (which are from a “first species”) that are entrapped or encapsulated in the hydrogel, the decellularized tissue will retain extracellular matrix proteins from the first species (that were produced by the “first species” stromal cells) and will also retain trace amounts of extracellular matrix protein sequence (or a fragment or variant thereof) from a second (distinct) species.

In other words, the decellularized tissue described herein may comprise extracellular matrix proteins from a first species and trace amounts of extracellular matrix protein sequence (or a fragment or variant thereof) from a second species, wherein the first species and second species are not the same. Examples of such tissues include tissues with human extracellular matrix proteins and trace amounts of porcine extracellular matrix protein sequence (or a fragment or variant thereof), or vice versa (i.e. tissues with porcine extracellular matrix proteins and trace amounts of human extracellular matrix protein sequence (or a fragment or variant thereof)). Other suitable combinations of cells and extracellular matrix protein sequence (or a fragment or variant thereof) may readily be identified by a person of skill in the art.

The extracellular matrix protein sequence (or a fragment or variant thereof) described herein may be also be synthetic. For example, the extracellular matrix protein sequence (or a fragment or variant thereof) that is present within the hydrogel may be synthetic. This results in a tissue comprising stromal cells and trace amounts of a synthetic extracellular matrix protein sequence (or a fragment or variant thereof.

In other words, the tissue described herein may comprise human stromal cells and trace amounts of synthetic extracellular matrix protein sequence (or a fragment or variant thereof). Other suitable combinations of cells and extracellular matrix protein sequence (or a fragment or variant thereof) may readily be identified by a person of skill in the art.

As used herein, the term “synthetic” refers to a product that is made by chemical synthesis, especially to imitate a natural product. The term is therefore used to describe extracellular matrix protein sequences (or fragments or variants thereof) that are not made by a cell. Synthetic amino acid sequences as also referred to as synthetic peptides herein. Methods for making synthetic peptides are well known in the art. Non-limiting examples include liquid phase peptide synthesis methods or solid peptide synthesis methods, e.g. solid peptide synthesis methods according to Merrifield, t-Boc solid-phase peptide synthesis, Fmoc solid-phase peptide synthesis, BOP (Benzotriazole-1-yl-oxy-tris-(dimethylamino)-phosphonium hexafluorophosphate) based solid-phase peptide synthesis, etc.

The peptides may include natural amino acids and/or synthetic amino acids. “Natural amino acids” are conventional amino acids defined by the genetic code, linked to each other by a normal peptide bond. By contrast, a “synthetic amino acid” is an amino acid that is not a conventional amino acid defined by the genetic code. Examples of synthetic amino acids are well known in the literature.

The peptides may be modified. In other words, the peptide may comprise amino acids modified by natural processes, such as post-translational maturation processes or by chemical processes, which are well known to a person skilled in the art. Such modifications are fully detailed in the literature. These modifications can appear anywhere in the peptide: in the peptide skeleton, in the amino acid chain or at the carboxy- or amino-terminal ends. Non-limiting examples of peptide modifications include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature. Accordingly, the terms “peptide”, “polypeptide”, “protein” may include for example lipopeptides, lipoproteins, glycopeptides, glycoproteins and the like. As a further non-limiting example, the peptide can be branched following ubiquitination or be cyclic with or without branching. This type of modification can be the result of natural or synthetic post-translational processes that are well known to a person skilled in the art.

As described elsewhere herein, the stromal cell tissues described herein may also be decellularized. Similar to the tissues described above, decellularized tissues may also comprise synthetic components. For example, when the stromal cell tissue (before decellularization) is generated using a hydrogel with synthetic extracellular matrix protein sequence (or a fragment or variant thereof), the decellularized tissue will retain trace amounts of the synthetic extracellular matrix protein sequence (or a fragment or variant thereof).

In other words, the decellularized tissue described herein may comprise human extracellular matrix proteins and trace amounts of synthetic extracellular matrix protein sequence (or a fragment or variant thereof). Other suitable combinations of cells and extracellular matrix protein sequence (or a fragment or variant thereof) may readily be identified by a person of skill in the art.

The methods described herein comprise the step of transferring/placing the hydrogel comprising entrapped or encapsulated stromal cells onto the curved surface. Alternatively or additionally, the methods described herein may comprise generating the hydrogel on the curved surface e.g. by extruding the hydrogel to cover a curved surface (e.g. using 3D printing).

When the method involves transferring/placing the hydrogel comprising entrapped or encapsulated stromal cells onto the curved surface, the hydrogel may be polymerised/gelled prior to being transferred onto the curved surface. Suitably, the hydrogel may be polymerised/gelled on a substantially flat surface prior to being transferred/placed onto the curved surface. In this context “a substantially flat surface” is a surface that has a curvature (in a concave or convex direction) of less than 0.03 mm⁻¹, less than 0.02 mm⁻¹, or less than 0.01 mm⁻¹, or less. Suitably, the substantially flat surface is a flat surface (i.e. has no curvature).

The hydrogel may be retained on a substantially flat surface for at least the length of time required for the hydrogel to polymerise/gel. It will be appreciated that the length of time required for the hydrogel to polymerise/gel will depend upon, for example, the properties of the hydrogel-forming polymer and/or environmental conditions (such as temperature and/or humidity). Merely by way of example, the hydrogel may be retained on a substantially flat surface for about 30 minutes or more, about 1 hour or more, about 2 hours or more, about 3 hours or more, about 4 hours or more, about 6 hours or more, about 9 hours or more, about 12 hours or more, about 24 hours or more, about 48 hours or more, about 72 hours or more. However, the inventors' believe that the effect of the curvature on cells entrapped or encapsulated in the hydrogel may be independent of the length of time the hydrogel is retained on a substantially flat surface. Accordingly, the hydrogel may be retained on a substantially flat surface for several days, weeks, months or longer.

The term “retaining the hydrogel on the curved surface” refers to keeping the hydrogel on the curved surface for the duration of step iii) of the method described herein.

The curved surface may be any suitable curved surface, for example it may be a concave surface or a convex surface. In other words, the hydrogel may be placed and retained on a concave (or convex) surface during the methods described herein. As described in detail in the examples section below, concave surfaces result in reduced cell flatness, high migration rates, increased nuclear circularity and high cellular alignment (relative to planar surfaces). This may be achieved without the use of an alignment-inducing substrate (for example a silk fibroin film). Accordingly, in some embodiments the method of making a transparent and curved stromal cell tissue does not comprise culturing of stromal cells on an alignment-inducing substrate (for example a silk fibroin film). Convex surfaces on the other hand result in increased cell flatness, high migration rates, reduced nuclear circularity and increased cell alignment (relative to planar surfaces). In some contexts, concave surfaces are preferred as they promote the highest levels of cell alignment.

In the methods described herein, the curved surface typically has a curvature (in a concave or convex direction) of from about 0.04 to about 0.5 mm⁻¹. Curvature of a surface is measured using standard means that are well known in the art (as described elsewhere herein). The curved surface may have a curvature of from 0.04 to about 0.5 mm⁻¹ (e.g. from about 0.1 to 0.4 mm⁻¹, particularly from about 0.2 to 0.3 mm⁻¹). The curvature of the surface determines the curvature of the stromal cell tissue (and subsequent decellularized tissue) that is generated by the methods described herein.

The curved surface may be formed from any appropriate material. Suitable materials may readily be identified by a person of skill in the art and include, for example, polystyrene, polyethylene, polyethylene terephthalate, polylactic acid, polycarbonate, acrylonitrile butadiene styrene, agarose, a hydrogel or glass surface. In a specific example, a borosilicate glass surface may be used.

In a particular example, the curved surface is formed from a hydrogel. In specific examples, the curved surface is formed from a hydrogel wherein the hydrogel comprises collagen.

For example, the stromal cell-containing hydrogel may be retained on the curved surface under appropriate cell culture conditions for at least two days, at least three days, at least four days etc until such time that a stromal cell tissue described elsewhere herein is generated.

The stromal cell-containing hydrogel is retained on the curved surface under appropriate cell culture conditions for at least twenty four hours. Appropriate cell culture conditions may be easily determined by a person of skill in the art and will depend on the type of stromal cells being used, the duration of cell culture etc.

For example, appropriate cell culture conditions may comprise retaining the hydrogel on the curved surface in cell culture medium. For example, the hydrogel may be immersed in cell culture medium whilst it is retained on the curved surface. The hydrogel may be partially immersed in the cell culture medium, but more typically, will be fully immersed in the cell culture medium.

The term “cell culture” as used herein refers to keeping the cells in an artificial environment under conditions favouring growth, differentiation, and/or continued viability of the cells. Cell growth may be promoted for example if cell number and/or cell viability is increased as compared to a suitable control.

The terms “cell culture medium” and “culture medium” (plural “media” in each case) refer to a nutritive solution for cultivating live cells and may be used interchangeably. The cell culture medium may be a complete formulation, i.e., a cell culture medium that requires no supplementation to culture cells, or may be an incomplete formulation, i.e., a cell culture medium that requires supplementation or may be a medium that may supplement an incomplete formulation or in the case of a complete formulation, may improve culture or culture results.

Various cell culture media will be known to those skilled in the art, who will also appreciate that the type of cells to be cultured may dictate the type of culture medium to be used.

Merely by way of example and not limitation, the culture medium may be selected from the group consisting of Dulbecco's Modified Eagle's Medium (DMEM), Ham's F-12 (F-12), Minimal Essential Medium (MEM), Basal Medium Eagle (BME), RPMI-1640, Ham's F-10, αMinimal Essential Medium (αMEM), Glasgow's Minimal Essential Medium (G-MEM), and Iscove's Modified Dulbecco's Medium(IMDM), or any combination thereof. Other media that are commercially available (e.g., from Thermo Fisher Scientific, Waltham, Mass.) or that are otherwise known in the art can be equivalently used in the context of this disclosure. Again, only by way of example, the media may be selected from the group consisting of 293 SFM, CD-CHO medium, VP SFM, BGJb medium, Brinster's BMOC-3 medium, cell culture freezing medium, CMRL media, EHAA medium, eRDF medium, Fischer's medium, Gamborg's B-5 medium, GLUTAMAX™ supplemented media, Grace's insect cell media, HEPES buffered media, Richter's modified MEM, IPL-41 insect cell medium, Leibovitz's L-15 media, McCoy's 5A media, MCDB 131 medium, Media 199, Modified Eagle's Medium (MEM), Medium NCTC-109, Schneider's Drosophila medium, TC-100 insect medium, Waymouth's MB 752/1 media, William's Media E, protein free hybridoma medium II (PFHM II), AIM V media, Keratinocyte SFM, defined Keratinocyte SFM, STEMPRO□ SFM, STEMPRO® complete methylcellulose medium, HepatoZYME-SFM, Neurobasal™ medium, Neurobasal-A medium, Hibernate™ A medium, Hibernate E medium, Endothelial SFM, Human Endothelial SFM, Hybridoma SFM, PFHM II, Sf 900 medium, Sf 900 II SFM, EXPRESS FIVE® medium, CHO-S-SFM, AMINOMAX-II complete medium, AMINOMAX-C100 complete medium, AMINOMAX-C140 basal medium, PUB-MAX™ karyotyping medium, KARYOMAX bone marrow karyotyping medium, and KNOCKOUT D-MEM, or any combination thereof.

The cell culture medium may be serum-free. For example, the serum-free medium may be DMEM or F-12, or a combination thereof.

The culture medium (for example DMEM or F-12, or a combination thereof) may further comprise a supplement. The supplement may be added at the start of step ii) of the method, or at any point thereafter. Supplements may be present in the cell culture medium throughout the method or may be added for a shorter period of time, as appropriate.

A supplement may be for example ascorbic acid, insulin, transferrin and/or sodium selenite. Other appropriate supplements include retinoic acid. In other words, retinoic acid may be added to the cell culture medium that is used in step ii) of the methods described herein. Supplementation of the cell culture medium with retinoic acid is advantageous because it enhances cell survival and extracellular matrix deposition in serum-free culture, reproducing the natural physiological state of stromal tissues.

The stromal cells within the hydrogel may be cultured in the presence of retinoic acid for a time period suitable to maintain cell viability and enhance deposition of specific extracellular matrix components. In one example, the cell may be cultured in the presence of retinoic acid for at least 0.5 days, at least 1 day, at least 2 days, at least 3 days, at least 4 days, at least 5 days, at least 6 days, at least 7 days, at least 8 days, at least 9 days, at least 10 days, at least 12 days, at least 15 days, at least 17 days, at least 21 days, at least 27 days, at least 30 days, or more. For example, the cell may be cultured in the presence of retinoic acid for at least 7 days, at least 15 days, at least 30 days, at least 45 days, at least 60 days, at least 75 days, or at least 90 days, or more. In one example the cells may be cultured in the presence of retinoic acid for a duration of from about 1 day to about 90 days, or from about 7 days to about 60 days.

By the same token, the cell may be cultured in the presence of retinoic acid, wherein the retinoic acid is at a concentration suitable to maintain cell viability and enhance deposition of specific extracellular matrix components. In one example, the cell may be cultured at a retinoic acid concentration of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, or more. For example, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM, or more. For example, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 950 μM, about 1000 μM or more.

Methods for assessing whether cell viability is maintained are well known in the art. Furthermore, methods for assaying for enhance deposition of specific extracellular matrix components are also known.

For example, the cell may be cultured in the presence of retinoic acid, wherein the retinoic acid is at a concentration from about 0.1 μM to about 1000 μM, from about 0.5 μM to about 750 μM, or from about 1 μM to about 500 μM. For example, the cell may be cultured in the presence of retinoic acid, wherein the retinoic acid is at a concentration from about 0.1 μM to about 10 μM or from about 0.5 μM to about 5 μM. Methods of determining a suitable concentration will be known to those skilled in the art.

Other appropriate supplements are known in the art, and include KTTKS lipopeptides. In other words, KTTKS lipopeptides may be added to the cell culture medium such that the cell culture medium that is used in step ii) of the methods described herein includes KTTKS lipopeptides in solution. Supplementation of the cell culture medium with KTTKS lipopeptides in solution is advantageous as it has been shown to increase collagen production in stromal cells.

The stromal cells within the hydrogel may be cultured in the presence of KTTKS lipopeptides for a time period suitable to increase the cell's collagen production. In one example, the cell may be cultured in the presence of the KTTKS lipopeptides for at least 1 hr, at least 2 hrs, at least 3 hrs, at least 4 hrs, at least 5 hrs, at least 6 hrs, at least 7 hrs, at least 8 hrs, at least 9 hrs, at least 10 hrs, at least 12 hrs, at least 14 hrs, at least 16 hrs, at least 18 hrs, at least 20 hrs, at least 22 hrs, at least 24 hrs, or more. For example, the cell may be cultured in the presence of the lipopeptides for at least 36 hrs, at least 48 hrs, at least 60 hrs, at least 72 hrs, at least 84 hrs, at least 96 hrs, at least 108 hrs, or at least 120 hrs, or more. In one example the cells may be cultured in the presence of KTTKS lipopeptide in solution for a duration of from about 1 hr to about 120 hrs, or from about 24 hrs to about 96 hrs.

By the same token, the cell may be cultured in the presence of KTTKS lipopeptides, wherein the lipopeptides are at a concentration suitable to increase the cell's collagen production. In one example, the cell may be cultured at an KTTKS lipopeptide concentration of about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 2 μM, about 3 μM, about 4 μM, about 5 μM, about 6 μM, about 7 μM, about 8 μM, about 9 μM, or more. For example, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM, or more. For example, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 950 μM, about 1000 μM or more.

For example, the cell may be cultured in the presence of the KTTKS lipopeptides, wherein the KTTKS lipopeptides are at a concentration from about 0.1 μM to about 1000 μM, from about 0.5 μM to about 750 μM, or from about 1 μM to about 500 μM. For example, the cell may be cultured in the presence of the lipopeptides, wherein the lipopeptides are at a concentration from about 0.1 μM to about 10 μM or from about 0.5 μM to about 5 μM. Methods of determining a suitable concentration will be known to those skilled in the art.

The term “lipopeptide” as used herein refers to an amphiphilic molecule comprising or consisting of a lipid portion and an amino acid portion. The terms “lipopeptide”, “amphiphilic molecule”, “peptide amphiphile” and “PA” are used interchangeably herein. The amphiphilic properties enable a plurality of lipopeptides to self-assemble into the supramolecular structure. Lipopeptides are well known and their self-assembly properties are well characterised in the art (see for example Cui H. et al., Biopolymers, 2010; 94 (1): 1-18). Appropriate lipopeptides may therefore easily be identified by a person of skill in the art e.g. by testing their propensity to self-assemble under certain conditions and form supramolecular structures. Lipopeptide self-assembly and corresponding c.a.c. can be evaluated by the Thioflavin (ThT) and pyrene (Pyr) fluorescence spectroscopy methods. Fluorescence spectra are recorded with a Fluorescence Spectrometer. For the ThT assay, the spectra are typically recorded from 460 to 600 nm using an excitation wavelength λₑₓ=440 nm and the lipopeptide dissolved in a 4-5×10⁻³% (w/v) ThT solution. For the Pyr assay, the spectra are typically recorded from 360 to 550 nm using an excitation wavelength λₑₓ=339 nm. Pyr assays are performed using a 1-1.5×10⁻⁵% (w/v) Pyr solution as a diluent. The Florescence intensity is plotted against a log of the lipopeptide concentration. The inflection point for the data denotes a change of environment for the ThT/Pyr molecule and is used to identify the c.a.c.

A KTTKS lipopeptide is a lipopeptide in which the amino acid portion comprises or consists of the amino acid sequence KTTKS or a fragment or variant thereof. An KTTKS fragment is a peptide that is shorter than the corresponding KTTKS amino acid sequence. An KTTKS fragment may share 100% identity with the portion of the KTTKS amino acid sequence that it corresponds to. The fragment may be at least 3 amino acid residues in length. For example, the fragment may be 3 or 4 amino acid residues in length. For example, the fragment may have a sequence selected from the group consisting of KTT, TTK, TKS, KTTK and TTKS. An KTTKS variant refers to an amino acid sequence in which one or more amino acids have been replaced by different amino acids as compared to the KTTKS amino acid sequence. For example, the variant may be selected from the group consisting of KTKTS, TTKKS, SKKTT, SKTET, TKSTK, or any one of the aforementioned variants wherein any one or more glutamate (E) residues is substituted with an aspartic acid (D) residue. In the context of the present disclosure, the fragment or variant may substantially retain the biological function of the corresponding sequence. For example, when the corresponding sequence is KTTKS, the fragment or variant may substantially retain the biological function of the KTTKS sequence.

The term “biological function” as used herein may refer to the ability to increase cell collagen production. This biological function is particularly relevant to KTTKS lipopeptides, as well as fragments or variants thereof. By “substantially retains” biological function, it is meant that the fragment or variant retains at least about 50%, 60%, 75%, 85%, 90%, 95%, 97%, 98%, 99%, or more, of the biological function of the corresponding KTTKS sequence. Indeed, the fragment or variant may have a higher biological function than the corresponding KTTKS sequence. The fragment or variant may have 110%, 120%, 130%, 140%, 150%, 160%, 170%, 180%, 190%, 200%, or more, of the biological function of the corresponding KTTKS sequence. The biological function may be, for example, the ability to increase collagen production in a cell. Methods of determining whether a fragment or variant has the ability to increase collagen production in a cell will be known to those skilled in the art. Merely by way of example, such examples include collagen staining, total collagen assay, or cell migration assay.

As used herein, the term “variant” refers to an amino acid sequence in which one or more amino acids have been replaced by different amino acids as compared to the corresponding amino acid sequence. It is well understood in the art that some amino acids may be changed to others with broadly similar properties without changing the nature of the activity of the peptide (conservative substitutions). Generally, the substitutions which are likely to produce the greatest changes in a peptide's properties are those in which (a) a hydrophilic residue (e.g., Ser or Thr) is substituted for, or by, a hydrophobic residue (e.g. Leu, lie, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

### Tissues

Tissues that are obtained or obtainable by the methods described above are also provided herein. Specifically, stromal cell tissues and corresponding decellularized tissues are described that are obtained or obtainable by these methods. The definitions provided above in the context of the methods apply equally to the tissues themselves.

The tissues described herein have a minimum thickness of at least 50 μm, a minimum visible light transmittance value of at least 0.5 and a curvature of from about 0.04 to about 0.5 mm⁻¹. The tissues also comprise:

a) stromal cells from a first species and

b) trace amounts of:
(i) an extracellular matrix protein sequence from a second species, or a fragment or a variant thereof; or
(ii) a synthetic extracellular matrix protein sequence or a fragment or a variant thereof.

In a particular example, the stromal cells of the tissue are from a first species and the extracellular matrix protein sequence is from a second (distinct) species.

In a particular example, the stromal cells in the tissue are human and the tissue comprises trace amounts of a synthetic or non-human extracellular matrix protein sequence, or a fragment or a variant thereof. In other words, in a particular example, the stromal cell tissue has a minimum thickness of at least 50 μm, comprises a trace amount of non-human or synthetic extracellular matrix protein sequence or a fragment or a variant thereof, has a minimum visible light transmittance value of at least 0.5 and a curvature of from about 0.04 to about 0.5 mm⁻¹, wherein the stromal cells are human. In one instance of this example, the human stromal cells may be corneal stromal cells such as corneal fibroblasts.

In a particular example, the tissue comprises trace amounts of collagen from a second species or a fragment or a variant thereof; or synthetic collagen. For example, the tissue may comprise trace amounts of synthetic or non-human collagen where the stromal cells are human stromal cells. In this context, the collagen fragment or a variant thereof may comprise or consist of an amino acid sequence selected from KTTKS (SEQ ID NO:2), GTPGPQGIAGQRGVV (SEQ ID NO: 12), GROGER (SEQ ID NO: 13), GLKGEN (SEQ ID NO: 14), GFOGER (SEQ ID NO: 15), and MNYYSNS (SEQ ID NO: 16) or a variant thereof.

In a particular example, the tissue may be in the form of a thin layer or disc.

In a particular example, the tissue has a thickness of from about 0.1 to 12.0 mm.

The stromal cell tissues described herein may be decellularized. Accordingly, a decellularized tissue obtainable or obtained by the methods described herein is also provided.

The decellularized tissues described herein have a minimum thickness of at least 50 μm, a minimum visible light transmittance value of at least 0.5 and a curvature of from about 0.04 and about 0.5 mm⁻¹. The decellularized tissues also comprise:

a) extracellular matrix protein sequences from a first species and

b) trace amounts of:
(i) an extracellular matrix protein sequence from a second species, or a fragment or a variant thereof; or
(ii) a synthetic extracellular matrix protein sequence or a fragment or a variant thereof.

In a particular example, the extracellular matrix proteins of the tissue are predominantly human and the tissue comprises trace amounts of a synthetic or non-human extracellular matrix protein sequence, or a fragment or a variant thereof. In other words, in a particular example, the decellularized tissue has a minimum thickness of at least 50 μm, comprises a trace amount of non-human or synthetic extracellular matrix protein sequence or a fragment or a variant thereof, has a minimum visible light transmittance value of at least 0.5 and a curvature of from about 0.04 to about 0.5 mm⁻¹, wherein the extracellular matrix proteins of the tissue are predominantly human. In this context, a tissue wherein the extracellular matrix proteins are “predominantly human” is a tissue wherein at least 90% (e.g. at least 91%, at least 92%, at least 93%, at least 94%, at least 95%, at least 96%, at least 97%, at least 98%, at least 99% etc) of the extracellular matrix proteins in the tissue are human extracellular matrix proteins.

In a particular example, the decellularized tissue comprises trace amounts of collagen from a second species or a fragment or a variant thereof; or synthetic collagen. For example, the decellularized tissue may comprise trace amounts of synthetic or non-human collagen where the tissue comprises predominantly human extracellular matrix proteins. In this context, the collagen fragment or a variant thereof may comprise or consist of an amino acid sequence selected from KTTKS (SEQ ID NO:2), GTPGPQGIAGQRGVV (SEQ ID NO: 12), GROGER (SEQ ID NO: 13), GLKGEN (SEQ ID NO: 14), GFOGER (SEQ ID NO: 15), and MNYYSNS (SEQ ID NO: 16) or a variant thereof.

In a particular example, the decellularized tissue may be in the form of a thin layer or disc.

In a particular example, the decellularized tissue has a thickness of from about 0.1 to 12.0 mm.

The tissues provided herein can be used to support epithelial growth. Accordingly, tissue composites are provided herein wherein the tissue composite is created from stromal cell tissues and an epithelial or endothelial cell layer. These composites may be comprised of stacked layers of the tissues with different mechanical properties/stiffness i.e. the upper or lower layer may be of a higher stiffness than the lower to support corneal epithelial and/or endothelial cell proliferation and function.

### Uses

The tissues described herein can be stored frozen and returned to culture, allowing for easy transportation and commercialization.

The tissues described herein have the ability to produce outgrowth when placed on a natural substrate (e.g. the cornea stroma). The tissues described herein are therefore useful for therapy, for example for altering corneal transparency and/or refractive properties in a subject. This may be used to replace the whole or partial (lenticules) donor corneal tissue.

The tissues described herein may individually be used for the purposes described herein, or may be used as a tissue composite. Tissue composites are described elsewhere herein. Accordingly, when referring to the use of a tissue herein, this encompasses the use of tissue composites (which comprise a tissue of the invention).

For example, the tissues may be used for intrastromal or onlay lamellar keratoplasty.

For onlay lamellar keratoplasty the tissue is sutured or glued onto the anterior of the cornea to change the anterior curvature and hence refractive properties. This may be performed with or without removal of host corneal stromal tissue (i.e. epikeratoplasty in which only epithelium is removed or deep lamellar keratoplasty in which the host tissue from the Descemet membrane up is removed and replaced with the tissue lens).

For intrastromal keratoplasty full or partial cuts (pockets, flaps) are made to the cornea and the tissue lens is inserted between the cut tissue. Intrastromal tissue lens may be positioned centrally (i.e. lamellar keratectomy) or peripherally (intracorneal ring).

As used herein, the term “subject” refers to any individual who may benefit from administration of the tissue, for example as part of a corneal transplant. Preferably, the subject is a mammal, most preferably a human.

The terms “treat”, “treating” or “treatment” refer to a clinical improvement of the relevant disease in a subject with the disease. Such a clinical improvement may be demonstrated by an improvement of the pathology and/or symptoms associated with the diseases. Symptoms associated with a corneal deficiency, disease or injury may include refractive error, corneal stromal dystrophies, Fuch's endothelial dystrophy, keratoconus, corneal ulcers, persistent epithelia defects, keratitis, corneal opacities, corneal burns, corneal abrasion, limbal stem cell deficiency, Bullous keratopathy.

The tissues may be part of a composition that further comprises a pharmaceutically acceptable diluent, carrier or excipient. The composition may further routinely contain pharmaceutically acceptable concentrations of salt, buffering agents, preservatives (for example antioxidants), supplementary immune potentiating agents such as adjuvants and cytokines and optionally other therapeutic agents.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients (such as the lipopeptides) of the composition. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

Excipients are natural or synthetic substances formulated alongside an active ingredient (e.g. the lipopeptides as provided herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Preservatives may be antioxidants. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

Cells used to generate the tissues described herein may be obtained from the subject to be treated, from a relative of the subject to be treated or from non-related donor. In some embodiments, the cells may be derived from a non-damaged eye from the subject to be treated.

Preferably, the cells are obtained from the same species as the subject.

The damaged ocular surface may be prepared by removing diseased cells and/or tissue. This may be done using standard surgical procedures, in order to expose the underlying corneal stroma. The tissue may then be placed onto or over the damaged ocular surface, e.g. onto or over the corneal stroma.

The hydrogel may be secured in place by appropriate means, e.g. using a therapeutic contact lens or inserting the tissue under the conjunctiva (the membrane surrounding the cornea), e.g. by first separating the conjunctiva from the sclera, then pulling the conjunctiva across the cornea and hydrogel gel. An appropriate suture, e.g. a purse string suture, may be used. The conjunctiva is now covering both the tissue and the cornea. A therapeutic contact lens might also be used to cover the conjunctiva.

Optionally, the tissue may be chemically bonded to the cornea e.g. by application of appropriate adhesive to the tissue or the cornea e.g. fibrin glue, cyanoacrylate, albumin or glutaraldehyde. Alternatively, the tissue may be chemically bonded to the cornea e.g. by the addition of riboflavin and UV light to form crosslinking between the collagen in the tissue and collagen in the cornea.

Optionally, the eyelid may be sutured closed to prevent infection and/or to maintain the position of the hydrogel, e.g. for 1 to 14 days.

The tissues described herein may also be used as in vitro tissue model, for example a model for understanding stromal cell interaction, a disease model for understanding corneal disease progression, or a screening model.

For example, a tissue described herein can be used as a screening model in vitro, for example for toxicity assays and in other applications. The tissues may also be suitable for investigating protein and gene expression of signalling molecules as well as global gene expression profiles under both normal and pathological conditions.

Further, such tissues can be used for preclinical research, pharmaceutical development, pharmaceutical and other testing, target gene therapy, toxicity testing, as a corneal disease model etc. Preclinical strategies related to corneal disease, tumour biology, scar formation etc., may also benefit from tissues as described herein.

For certain applications, the tissues may be formed by using cells from diseased tissues. For example, tissues can be formed using stromal cells from diseased corneal tissue. Diseased corneal tissue includes corneal diseases such as corneal ectasia, corneal stromal dystrophies, limbal stem cell deficiencies, persistent epithelial defects, Fuchs' endothelial dystrophy, Epithelial basement membrane dystrophy.

For example, abnormal tissues which reflect clinical corneal diseases can be established by using stromal cells from clinical patients. The tissue can then be compared with those generated from normal stromal cells, which aids in studying the molecular and gene profile underlying the formation of diseases and in developing gene therapies.

Disease models using the tissues described herein can also be used in pharmaceutical testing or pre-clinical treatment for relevant corneal diseases. Such tissues provide an alternative and much improved tool to animal disease models and 2D cell culture systems.

The tissues described herein may be used to observe the effects of agents, e.g therapeutic agents, on organs, i.e. diseased tissues, and to identify agents that are capable of treating diseases, and reducing the symptoms thereof.

Accordingly, a method of screening compounds to identify agents useful for treating diseases, in particular corneal diseases is also provided herein.

The method comprises providing a tissue of the invention (generated using non-diseased stromal cells, diseased stromal cells, or a combination thereof), contacting it with a test compound, and determining the effect of the test compound on the tissue.

The test compounds are preferably administered to the tissue in an amount sufficient to and for a time necessary to exert an effect upon said tissue. These amounts and times may be determined by a person of skill in the art using standard procedures known in the art. Parameters that may be measured as an indication of the effect of the test compound may include, for example, changes in cell integrity, changes in cell viability, and/or structural changes within the tissue.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1

### Effects of Surface Curvature on Corneal Stromal Cell Behaviour

### Introduction

Studies characterising the effect of curvature as a surface cue influencing the migration and alignment of different cell types have largely been restricted to substrate diameters at the nano- and microscales (Ø=1*10⁻⁷-10⁻⁴ m, respectively) with corresponding curvatures (k=1/R). Studies using surfaces on spheroids showed that concave curvatures with Ø=0.5 mm promoted more persistent migration, alignment and altered attachment morphology in human mesenchymal stem cells (MSCs) compared to surfaces exhibiting convex curvatures of corresponding diameter (Werner et al., 2017). Previously, the inventors also showed that tissues formed by corneal stromal cells migrating from flat to convex surfaces the average size of the human cornea (i.e., Ø=13 mm, k=0.154 mm⁻¹) were able to align and deposit collagen type I fibrils with significantly higher organisation compared to planar controls (Gouveia et al., 2017). Furthermore, cells grown onto such curved surfaces were shown to express significantly higher levels of corneal-characteristic crystallins and proteoglycans. Together, these studies posit that cell behaviour may be modulated by curved geometries many times larger than the size of a cell.

The inventors have now further investigated the effect of surface curvature on the behaviour of corneal stromal cells, and particularly the range of curvatures capable of promoting the self-organisation of this cell type into highly-aligned cultures.

### Methods

### Cell Isolation and Culture

Cells were extracted from corneal limbal rings of three healthy human donors, as previously described (Gouveia et al., 2017). Corneal stromal cells were then expanded in vitro with DMEM/F-12 supplemented with 5% FBS and 1% (w/v) penicillin/streptomycin. Cells expanded in this medium showed an elongated, spindle-shaped morphology characteristic of stromal fibroblasts. Prior to the experiments, cells no older than passage 4 were incubated for 3 days in serum-free medium (DMEM/F12 supplemented with 1% (w/v) Insulin-Transferin-selenium, 1 mM L-ascorbic acid 2-phosphate, and 1% (w/v) penicillin/streptomycin). Such cells assumed dendritic shape morphologies reminiscent of the quiescent corneal stromal cells found in vivo.

### Substrate Preparation and Seeding

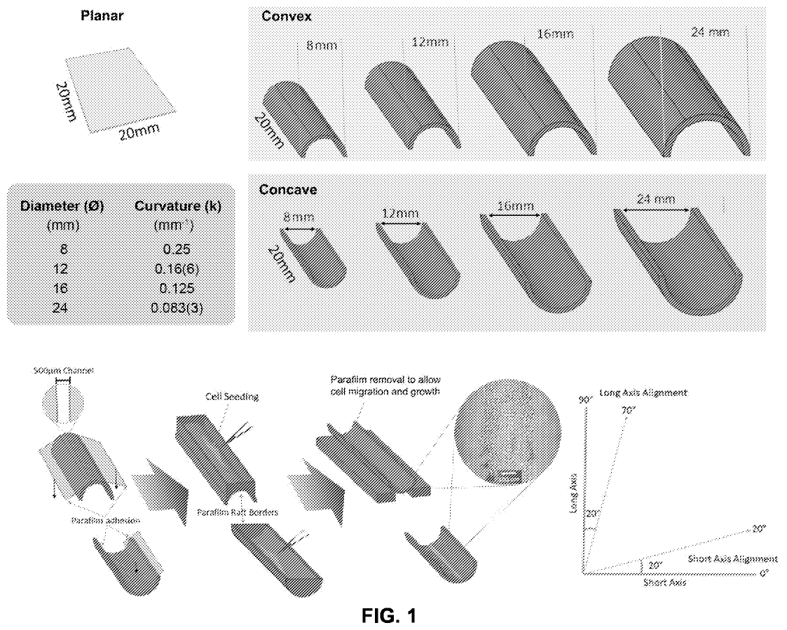

Borosilicate glass semi-cylinders with different diameters were custom-made to test the effect of different surface curvatures on cell behaviour (FIG. 1). Normal glass coverslips 20×20mm were used as planar control surfaces (FIG. 1). All substrates were sterilized by autoclave. Subsequently, surfaces were covered with Parafilm, using this silicone wrap as a ‘mask’ for limiting the initial cell seeding area to a defined, 500 μm-wide longitudinal line across the substrates (FIG. 1). Serum-starved cells were then suspended using TrypLE enzyme digest for 5 minutes at 37° C., centrifuged for enzyme removal, re-suspended in serum-free medium at 1×10⁵ cells/ml, and seeded over the exposed surface of the glass substrates (0.5 mL per surface) (FIG. 1). After 2 h incubation at 37° C., all Parafilm wrap was removed aseptically with tweezers, exposing the remaining glass surface to cell migration and growth over the long (longitudinal) and short (arc) axis of the surface (FIG. 1). To this purpose, substrates were maintained fully immersed in serum-free medium for 4 days, with medium replaced every other day.

### Cell Migration

Quantitative microscopy was performed using a Leica DM IL LED Tissue Culture Microscope to track cell migration over a period of 3 days (T0, T1, T2, T3) with TO corresponding with initial cell seeding. Images were taken every 24 hours and multifocal image stacks were processed utilizing the ImageJ Extended Depth of Field plugin. Images were then segmented into 40,000 μm² grids with migration of cell populations along the substrate short axis tracked by means of line analysis across these grids where a positive value would return at >20% cell saturation per grid. A sampling size of 10 data points at set coordinates corresponding to distance of migration along the short axis were taken per field of view (FOV) (2100×2100 μm), and 3 FOV images were taken per sample per day. Repeated imaging of set FOVs in each sample was ensured by digitally verifying surface coordinates of each image taken in relation to a marked ‘leading’ edge on each semi-cylinder and coverslip. Imaging multifocal error on curved surfaces was corrected for by subjecting each extrapolated migration distance value to curved surface correction using the equation [(2πR)*((((sin\hat{ }(−1) ((Dist/2)/R)*2)))/360)], to maintain accuracy across differential curvature (k) geometries.

### Nuclear Morphology

Nuclear shape was characterized with quantitative fluorescence microscopy. Cells were fixed in 4% paraformaldehyde for 20 min at room temperature on day 4 (T4) and subsequently blocked with 2.5% BSA in phosphate buffer solution, and then stained with Bisbenzimide Hoechst 33392 DNA intercalating stain (350/461 nm) and Alexa Fluor 594 phalloidin (581/609 nm) for 2 h. Images taken at λ=461 nm were binarised and analysed for circularity using the equation (4π×([Area])/([Perimeter]2)) in ImageJ to give a range of frequencies and circularity values ranging between 0 and 1, with the former being the least circular and the later a perfect circle. Three FOV samples were taken per substrate per experiment.

### Cell Orientation

Cell orientation/alignment was characterised via the analysis of f-actin fluorescence micrographs. Briefly, cells stained with Alexa Fluor 594 phalloidin were imaged at λ=609 nm and micrographs analysed with ImageJ plugin OrientationJ, which converts the fluorescence-marked cellular structures into a vector map. Vectors were then grouped in terms of preferential orientation (between 0 and 90°, corresponding to the short and long axis, respectively), and vector frequency expressed in 5° bins. Cells were considered aligned when showing preferential actin orientation (within 20°) relative to the short or the long axis (FIG. 1).

### Statistical Analysis

Statistical analysis was performed using Graphpad Prism 7.0. Cell migration data was subject to parametric ANOVA tests with further comparisons analysed with Dunnett's multiple comparisons test, using 30 data points from 3 FOVs collected from each substrate sample per day. Daily migration data from each sample was then averaged and standard deviation taken. Mean migration rate was calculated from the average migration across all days. Mean migration profiles were then compared between substrates to determine if significant differences arose between total migration (final migration width at T3 expressed as a percentage of initial seeding at T0), or migration rate (μm/hour) when comparing convex and concave surfaces at differential curvatures (k) and orientations. Frequencies of alignment vectors between 0 and 90° were potted as a % of the total number of vectors. Frequency data was averaged across 3 experiments and subsequently subjected to parametric one-way ANOVA tests and further comparisons with the Sidak's multiple comparisons test. Planar surfaces were used to determine the assumed threshold at which no cell alignment is observed. Circularity data was not normally distributed and thus was subjected to non-parametric tests. A sampling size of 50 data points per sample per experiment was taken and plot into a dataset combining data from each sample across all 3 experiments. A Kruskal-Wallis test with Dunn's multiple comparisons test was employed between all samples following the Mann-Whitney's test to determine if significant differences in nuclear circularity occurred across all substrates.

### Results

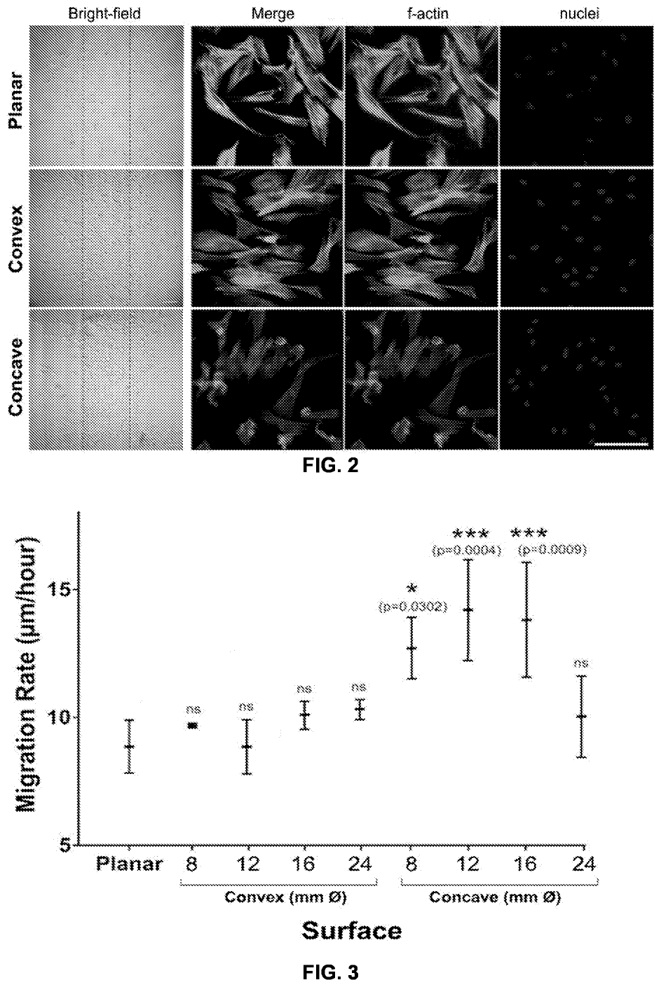

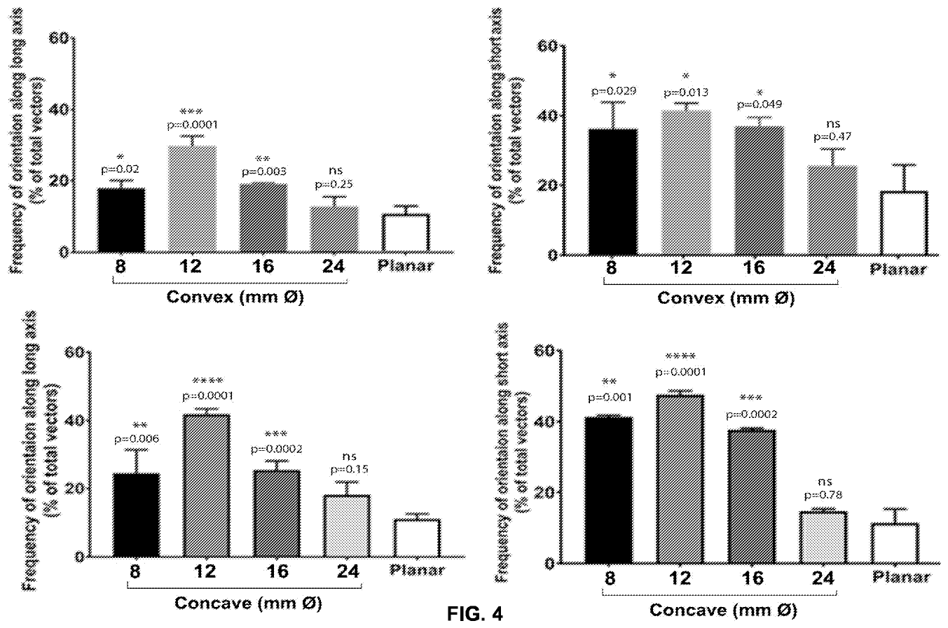

Confocal microscopy studies in microscale convex structures have previously shown that cell bodies are pulled ‘upwards’ minimizing contact with the substrate to distinct adhesion points. Consequently, the inventors qualitatively observed that cells were less flat across all concave substrate surfaces compared to convex substrates where cells are seen to adopt a ‘stretched’ morphology and appeared flatter under the microscope (FIG. 2). Increased cell surface area in convex surfaces can be explained by a physical model where surface tension between attachments points (focal adhesions) placed along such sloped surface pull the cell in an outward fashion thus flattening the cell. Consequently, cells need to remodel a larger contact area during movement leading to slower migration. Agreeing with this model, the inventors observed corneal cells migrating significantly faster on concave surfaces compared to convex surfaces (FIGS. 2 and 3). The migration rate seen in planar samples closely replicates that of the rate seen in previous studies (Fernandez-Perez et al., 2019). These results reflect the chord physical model where cells are defined as tensile elements of finite viscoelastic nature and stretched upwards between focal adhesions on convex surfaces and pulled down towards the surface on concave structures. For human MSCs, the upward curvature threshold for the differential lift-off effect was determined to be Ø=0.75 mm (Werner et al., 2017). The inventors have now observed that corneal stromal cells manifest the same behaviour but at meso-scale curvatures up to Ø=16 mm (FIG. 3). During cell migration, cells extend protrusions such as lamellipodia and filopodia forming adhesions to the substrate surfaces subsequently retracting their tail end. Following this model of migration, cells polarize and act in anisotropic manners during migration to effectively convert contractile action into motion. The inventors therefore found that both concave and convex surfaces significantly encouraged higher total cell alignment (along both transversal and arc axes) compared with planar controls (FIG. 4). However, concave surfaces up to Ø=16 mm promoted the highest degree of cell orientation, with an optimum at Ø=12 mm (FIG. 4).

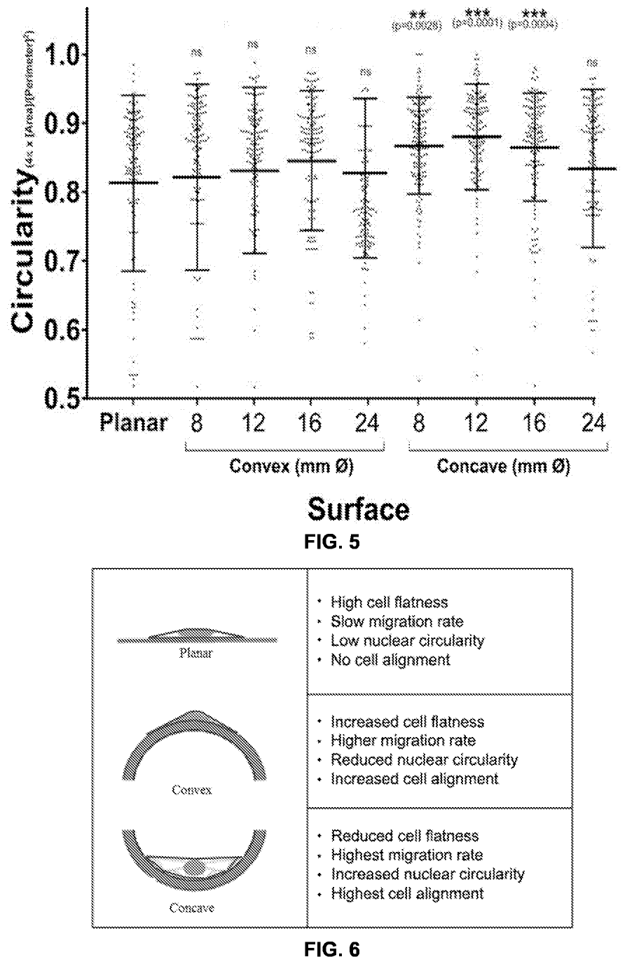

With increasing surface area, tensioned actin cytoskeletal elements also transmit forces through LINC complexes into the nucleus. Decreased nuclear circularity on convex surfaces suggests high intracellular tension is exerted upon the nucleus. Accommodating the consequent elastic and compressive stresses acting upon the nucleus, increased nuclear deformation ensues, with cells grown upon a convex surface expressing an increased surface area as a result of increased surface tension. In accordance with these notions, the inventors observed higher nuclear circularity in cells on concave surfaces up to Ø=24 mm (FIG. 5). The results show a positive correlation between nuclear circularity and concave diameter. These findings suggest that through measuring nuclear circularity as a product of nuclear deformation the inventors are able accurately assume the intracellular stress state. Also of particular interest is that circularity observations were found to be more variable in convex compared to concave substrates. This alongside the lower alignment on convex substrates may suggest that differential orientation may modulate the way cells interact with and adhere along single axis curvatures and thus alter the compressive forces experienced by the nucleus. The inventors thus conclude that curvatures even in scales significantly larger than the cell's diameter are able to differentially regulate cell migration rate, morphology, nuclear circularity, and alignment (FIG. 6).

### Example 2

### Effects of Surface Curvature on Gene Expression and Transparency Between Cells Within Hydrogels

### Methods

### Collagen Gel Production and Culture

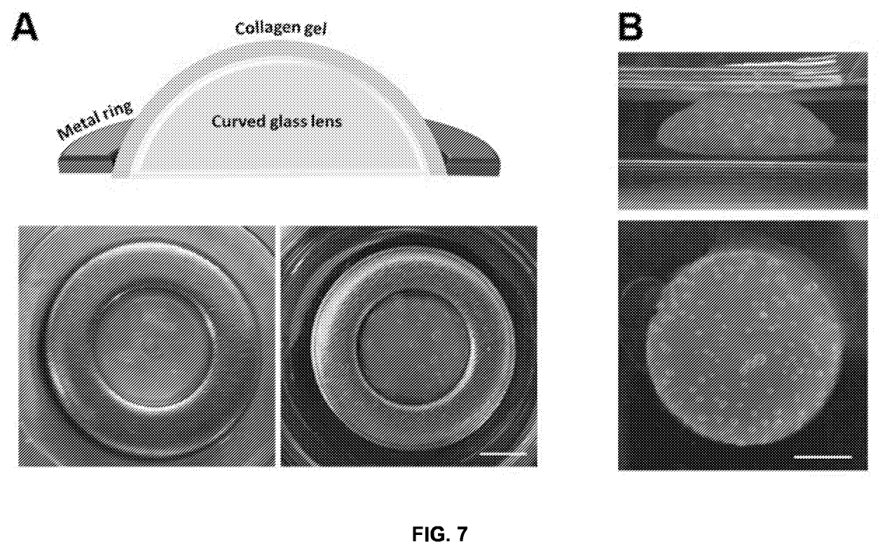

Ice-cold collagen type-I solution (2 g/L, extracted in 0.6% acetic acid from rat tail; First Link Ltd, UK) was mixed with 10× Modified Essential Medium (Life Technologies, USA) and neutralised with 1M NaOH at a 1:7:1 volume ratio. Human corneal stromal cells were added to this mix at 5-20×10⁴ cells/ml, and homogenised by gentle agitation for 30 s. The cell-containing mix was then pipetted into 24 well-plates (1 mL/cm²) and allowed to solidify at 37° C. for 30 min. Absorbent inserts (Lonza, Switzerland) were applied to compress the gels into discs of collagen with a diameter of 13-16 mm. The resulting 3D collagen gels were subjected to curvature cues by gently transfer onto glass domes 3 mm high and 10 mm diameter, using a metal washer to hold them in place (FIG. 7a), and cultured in serum-free media for up to 7 days. Planar gels were used as negative controls.

### Gene Expression Analysis

Curved and planar collagen gels were homogenized using 1 mL Trizol and a syringe, supplemented with 200 μl of chloroform, and allowed to rest for 15 min at room temperature. After phase separation, the mix was centrifuged at 18,000×g for 15 min. The top phase containing mRNA was collected, suspended in ethanol, and processed using the RNeasy Mini Kit (Qiagen, UK) according to manufacturer specifications. The mRNA was then treated with DNase to eliminate genomic DNA. Quality assessment of the RNA was performed using a Nanodrop 2000 spectrophotometer (Thermo Scientific, UK) for quantification and purity. The RNA was reverse-transcribed with the RT2 First Strand kit (Qiagen, UK) according to manufacturer specifications in a TC-Plus thermocycler (Techne, UK), and the resulting cDNA used for qPCR using direct dye binding in the Eco Real-Time PCR System (Illumina, USA), with the appropriate primers (see SEQ ID Nos: 24 to 53 below) and using 40 cycles of 10 s denaturation at 95° C., 30 s annealing at 60° C. and 15 s extension at 72° C. Data corresponded to mean±SD of 3 independent experiments with expression of each gene of interest normalized to housekeeping gene GAPDH.

### Optical Properties Analysis

3D collagen gels were fixed in 4% paraformaldehyde for 30 min, washed thrice with PBS, and maintained immersed in PBS for analysis. The Inverse Adding Doubling (IAD) method was used to determine scattering and absorption coefficients of the curved and planar gels, as well as native porcine cornea (positive control), by means of total reflection and total transmission measurement with integrating spheres. Samples were placed between two glass slides and measurements were taken in the centre of the sample at λ=457.9, 488, 514.5 nm (Argon Ion Laser-Stellar-Pro-L Model, Modu-Laser; USA), and 632.8 nm (He—Ne laser-30564 Model, Research Electro-Optics; USA). Curved 3D collagen gels and corneas were radially cut to obtain a flattened, mountable sample. All experiments were performed in duplicate.

### Results

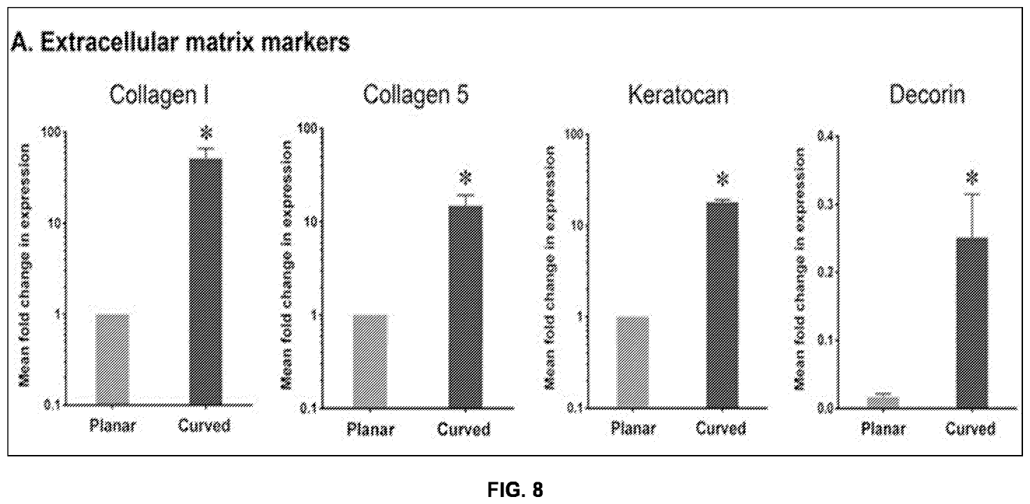

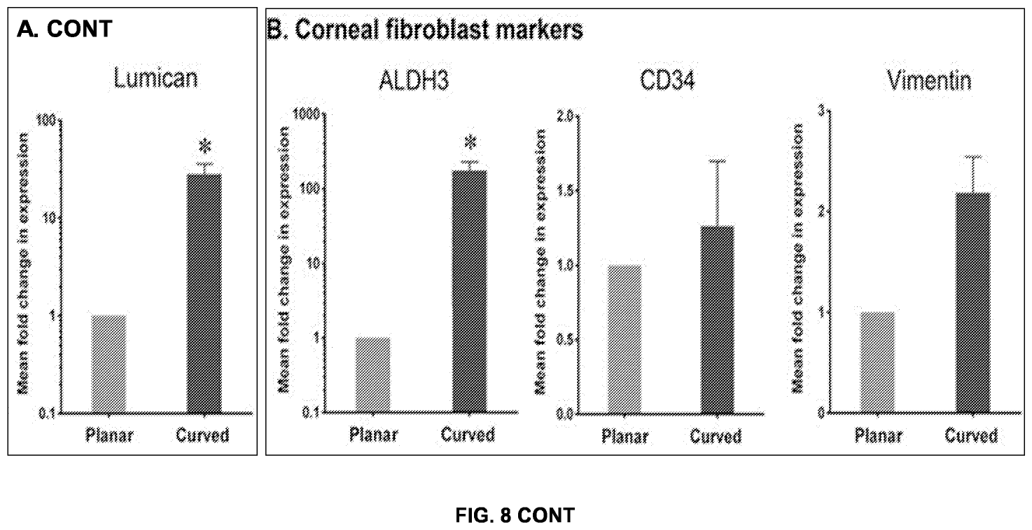

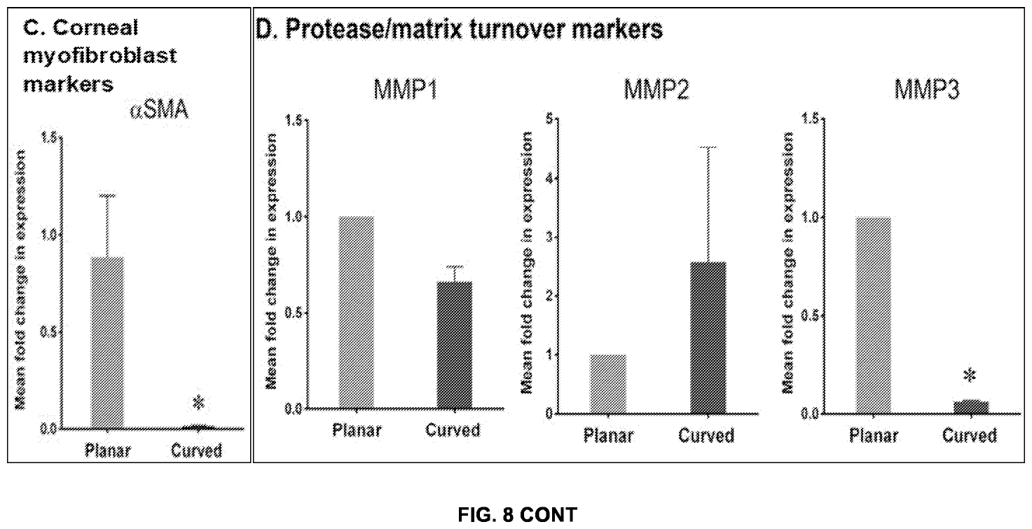

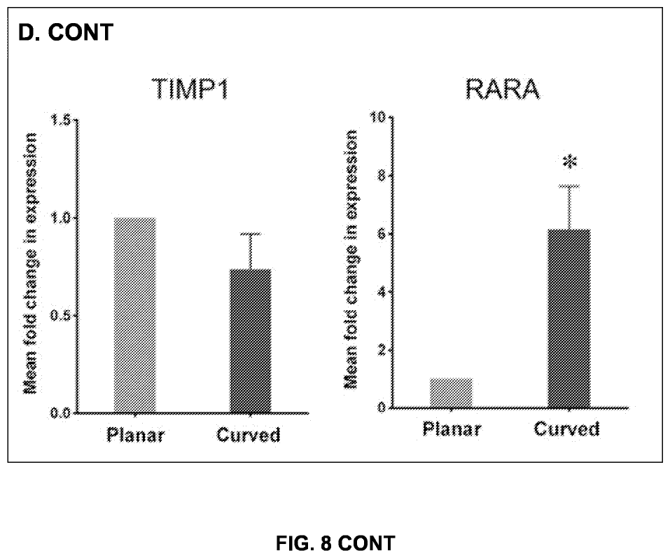

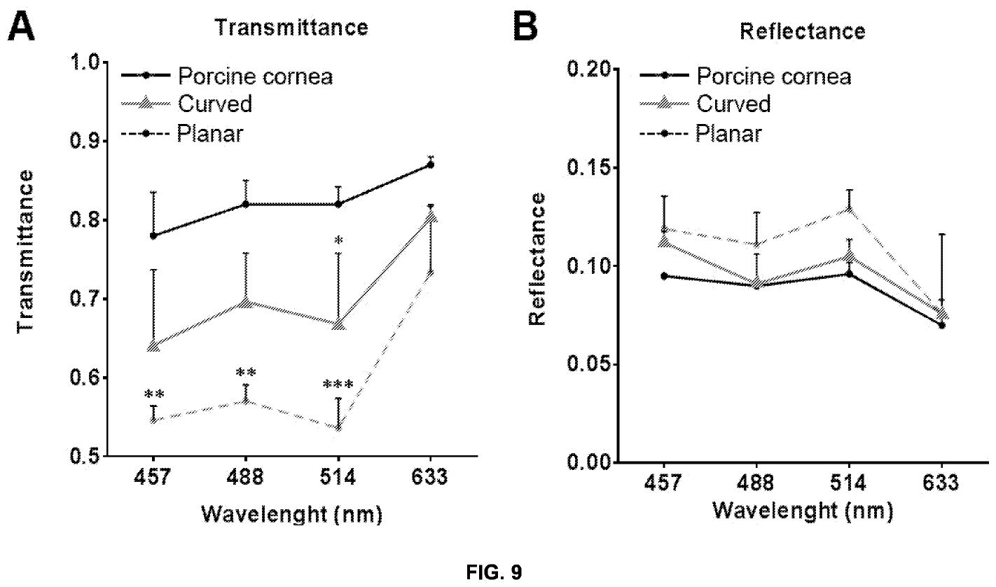

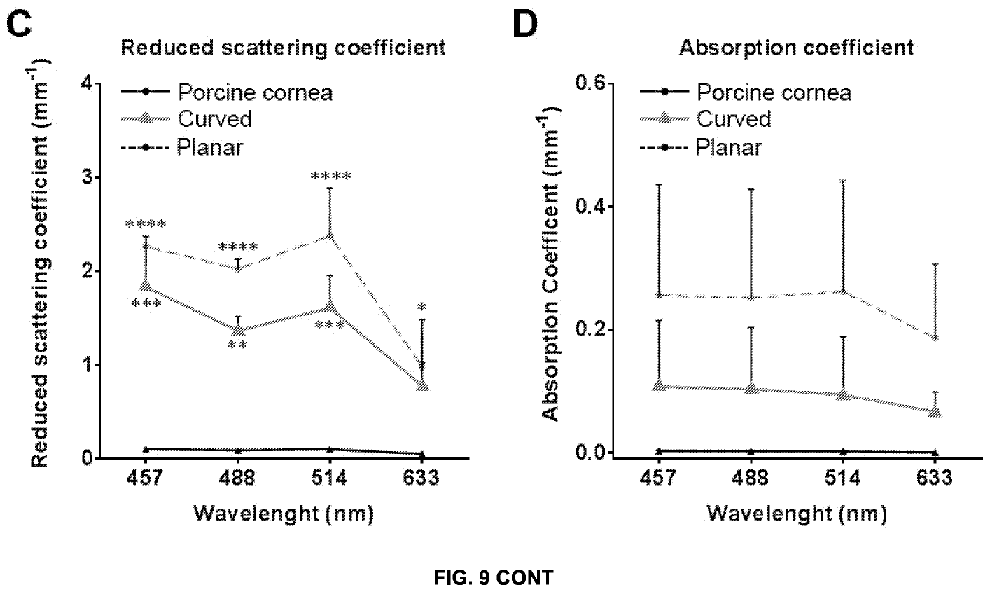

Curvature cues at meso/macroscales also influence cell phenotype in 3D environments. This was demonstrated by encapsulating corneal stromal cells within compressed collagen gels, moldable 3D scaffolds able to be induced into dome-shaped curvatures with defined diameters (FIG. 7). Cells subjected to such curvature changed their gene expression profile, with upregulation of several markers characteristic of the corneal extracellular matrix (FIG. 8a) and resident stromal cell population (FIG. 8b). In particular, cells within curved 3D collagen gels expressed significantly higher transcript levels for the cornea-specific proteoglycan keratocan, as well as for the ALDH3 crystallin compared to those within planar gels. These molecules have been previously described as fundamental for assuring corneal transparency. In contrast, cells within curved 3D collagen gels showed reduced expression of wound response/stress markers strongly associated with corneal opacification/haze (FIGS. 8c and d). Together with possible alterations in collagen fibril organisation and gel ultrastructure induced by the curvature method, these changes in cell behaviour may also explain the increased transparency of curved 3D collagen gels compared to their planar counterparts (FIG. 9).

Amino acid sequences:
Fibronectin:
SEQ ID NO: 1
RGD
SEQ ID NO: 5
RGDS
SEQ ID NO: 6
PHSRN
SEQ ID NO: 7
LDVP
SEQ ID NO: 8
WQPPRARI
SEQ ID NO: 9
IGD
SEQ ID NO: 10
REDV
SEQ ID NO: 11
IDAP
Collagen:
SEQ ID NO: 2
KTTKS
SEQ ID NO: 12
GTPGPQGIAGQRGVV
SEQ ID NO: 13
GROGER
SEQ ID NO: 14
GLKGEN
SEQ ID NO: 15
GFOGER
SEQ ID NO: 16
MNYYSNS
Lumican:
SEQ ID NO: 3
YEALRVANEVTLN
SEQ ID NO: 17
EVTLN
SEQ ID NO: 18
ELDLSYNKLK
Laminin:
SEQ ID NO: 1
RGD
SEQ ID NO: 19
YIGSR
SEQ ID NO: 20
IKVAV
SEQ ID NO: 21
CCRRIKVAVWLC
SEQ ID NO: 22
HSNGLPGGGSEEEAAAVVV
SEQ ID NO: 23
HSNGLPLGGGSEEEAAAVVV(K)
SEQ ID NO: 4
HSNGLPLGGGSEEEAAAVVVK
Nucleic acid sequences:
Primers:
Collagen 1
SEQ ID NO: 52
F: CCTGCGTGTACCCCACTCA
SEQ ID NO: 53
R: ACCAGACATGCCTCTTGTCCTT
Collagen 5
SEQ ID NO: 24
F: ATCTTCCAAAGGCCCGGATG
SEQ ID NO: 25
R: AAATGCAGACGCAGGG-TACA
Vimentin
SEQ ID NO: 26
F: CCTCCTACCGCAGGATGTT
SEQ ID NO: 27
R: CTGTAGGTGCGGGTGGAC
Decorin
SEQ ID NO: 28
F: CTGCTTGCACAAGTTTCCTG
SEQ ID NO: 29
R: GACCACTCGAAGATGGCATT
Keratocan
SEQ ID NO: 30
F: TATTCCTGGAAGGCA-AGGTG
SEQ ID NO: 31
R: ACCTGCCTCACACTTCTAGACC
Lumican
SEQ ID NO: 32
F: CCTGGTTGAGCTGGATCTGT
SEQ ID NO: 33
R: TAGGATGGCCCCAGGA
ALDH3
SEQ ID NO: 34
F: CCCCTTCAACCTCACCATCC
SEQ ID NO: 35
R: GTTCTCACTCAGCTCCGAGG
MMP1
SEQ ID NO: 36
F: AGGTCTCTGAGGGTCAAGCA
SEQ ID NO: 37
R: CTGGTTGAAAAGCATGAGCA;
SMA
SEQ ID NO: 38
F: CTGAGCGTGGCTATTCCTTC
SEQ ID NO: 39
R: TTCTCAAGGGAGGATGAGGA
MMP2
SEQ ID NO: 40
F: CCAAGCGTCTAGCAATACC
SEQ ID NO: 41
R: TCTGGGGCAGTCCAAAGAAC
MMP3
SEQ ID NO: 42
F: TGCTTTGTCCTTTGATGCTG
SEQ ID NO: 43
R: AAGCTTCCTGAGGGATTTGC;
TIMP1
SEQ ID NO: 44
F: ATTGCTGGAAAACTGCAGGAT
SEQ ID NO: 45
R: TCCACAAGCAATGAGTGCCA
RARA
SEQ ID NO: 46
F: AGTCCTCAGGCTACCACTAT
SEQ ID NO: 47
R: CCTCCTTCTTCTTCTTGTTT
CD34
SEQ ID NO: 48
F: CTTGGGCATCACTGGCTATT
SEQ ID NO: 49
R: TCCACCGTTTTCCGTGTAAT
GAPDH
SEQ ID NO: 50
F: AGCCGAGCCACATCGCTGAG
SEQ ID NO: 51
R: TGACCAGGCGCCCAATACGAC

### REFERENCE LIST

### Patent Literature

WO2006/003442

WO2007/060459

WO2009/004351

### Non-Patent Literature

Fagerholm, P., N. S. Lagali, J. A. Ong, K. Merrett, W. B. Jackson, J. W. Polarek, E. J. Suuronen, Y. Liu, I. Brunette and M. Griffith (2014). “Stable corneal regeneration four years after implantation of a cell-free recombinant human collagen scaffold.” Biomaterials 35 (8): 2420-2427.

Fernández-Pérez, J. and M. Ahearne (2019). “Influence of Biochemical Cues in Human Corneal Stromal Cell Phenotype.” Current Eye Research 44 (2): 135-146.

Ghezzi, C. E., J. Rnjak-Kovacina and D. L. Kaplan (2015). “Corneal tissue engineering: recent advances and future perspectives.” Tissue Eng Part B Rev 21 (3): 278-287.

Gouveia, R. M. and C. J. Connon (2016). 7—Collagen scaffolds for corneal regeneration. Biomaterials and Regenerative Medicine in Ophthalmology (Second Edition). T. V. Chirila and D. G. Harkin, Woodhead Publishing: 151-177.

Gouveia, R. M., E. Koudouna, J. Jester, F. Figueiredo and C. J. Connon (2017). “Template Curvature Influences Cell Alignment to Create Improved Human Corneal Tissue Equivalents.” Advanced Biosystems 1 (12): 1700135.

Hassell, J. R. and D. E. Birk (2010). “The molecular basis of corneal transparency.” Experimental Eye Research 91 (3): 326-335.

He, X. and Y. Jiang (2017). “Substrate curvature regulates cell migration.” Phys Biol 14 (3): 035006.

Levis, H. J., J. Menzel-Severing, R. A. Drake and J. T. Daniels (2013). “Plastic compressed collagen constructs for ocular cell culture and transplantation: a new and improved technique of confined fluid loss.” Curr Eye Res 38 (1): 41-52.

Li, F., D. Carlsson, C. Lohmann, E. Suuronen, S. Vascotto, K. Kobuch, H. Sheardown, R. Munger, M. Nakamura and M. Griffith (2003). “Cellular and nerve regeneration within a biosynthetic extracellular matrix for corneal transplantation.” Proc Natl Acad Sci USA 100 (26): 15346-15351.

Li, Q., A. Kumar, E. Makhija and G. V. Shivashankar (2014). “The regulation of dynamic mechanical coupling between actin cytoskeleton and nucleus by matrix geometry.” Biomaterials 35 (3): 961-969.

Li, S., Y. Han, H. Lei, Y. Zeng, Z. Cui, Q. Zeng, D. Zhu, R. Lian, J. Zhang, Z. Chen and J. Chen (2017). “In vitro biomimetic platforms featuring a perfusion system and 3D spheroid culture promote the construction of tissue-engineered corneal endothelial layers.” Sci Rep 7 (1): 777.

Lwigale, P. Y. (2015). Chapter Four—Corneal Development: Different Cells from a Common Progenitor. Progress in Molecular Biology and Translational Science. J. F. Hejtmancik and J. M. Nickerson, Academic Press. 134: 43-59.

McMonnies, C. W. and G. C. Boneham (2007). “Corneal curvature stability with increased intraocular pressure.” Eye Contact Lens 33 (3): 130-137.

Rafat, M., F. Li, P. Fagerholm, N. S. Lagali, M. A. Watsky, R. Munger, T. Matsuura and M. Griffith (2008). “PEG-stabilized carbodiimide crosslinked collagen-chitosan hydrogels for corneal tissue engineering.” Biomaterials 29(29): 3960-3972.

Werner, M., S. B. G. Blanquer, S. P. Haimi, G. Korus, J. W. C. Dunlop, G. N. Duda, D. W. Grijpma and A. Petersen (2017). “Surface Curvature Differentially Regulates Stem Cell Migration and Differentiation via Altered Attachment Morphology and Nuclear Deformation.” Advanced Science 4 (2): 1600347.

Wu, J., J. Rnjak-Kovacina, Y. Du, M. L. Funderburgh, D. L. Kaplan and J. L. Funderburgh (2014). “Corneal stromal bioequivalents secreted on patterned silk substrates.” Biomaterials 35 (12): 3744-3755.

Zhang, W., J. Chen, L. J. Backman, A. D. Maim and P. Danielson (2017). “Surface Topography and Mechanical Strain Promote Keratocyte Phenotype and Extracellular Matrix Formation in a Biomimetic 3D Corneal Model.” Adv Healthc Mater 6 (5).

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A method of making a transparent and curved stromal cell tissue having a minimum thickness of at least 50 μm, the method comprising:
i) encapsulating or entrapping stromal cells in a hydrogel, wherein the hydrogel comprises cell adhesion motifs;
ii) transferring the hydrogel onto a curved surface; and
iii) retaining the hydrogel on the curved surface under appropriate cell culture conditions for at least twenty four hours to generate a curved stromal cell tissue with a minimum visible light transmittance value of at least 0.5, wherein the curved surface has a curvature of from about 0.04 to about 0.5 mm⁻¹.

2. The method of claim 1, wherein the cell adhesion motif is an extracellular matrix protein sequence or a fragment or a variant thereof.

3. The method of claim 2, wherein the extracellular matrix protein is selected from the group consisting of fibronectin, collagen, lumican, decorin, laminin, vitronectin, fibrinogen, elastin, biglycan, heparin, tenascin and osteopontin.

4. The method of claim 3, wherein the cell adhesion motif is:
a) a fibronectin fragment comprising or consisting of an amino acid sequence selected from RGD (SEQ ID NO: 1), RGDS (SEQ ID NO: 5), PHSRN (SEQ ID NO: 6), LDVP (SEQ ID NO: 7), WQPPRARI (SEQ ID NO: 8), IGD (SEQ ID NO: 9), REDV (SEQ ID NO: 10), and IDAP (SEQ ID NO: 11) or a variant thereof;
b) a collagen fragment comprising or consisting of an amino acid sequence selected from KTTKS (SEQ ID NO:2), GTPGPQGIAGQRGVV (SEQ ID NO: 12), GROGER (SEQ ID NO: 13, GLKGEN (SEQ ID NO: 14), GFOGER (SEQ ID NO: 15), and MNYYSNS (SEQ ID NO: 16) or a variant thereof; or
c) a lumican fragment comprising or consisting of an amino acid sequence selected from EVTLN (SEQ ID NO: 17), ELDLSYNKLK (SEQ ID NO: 18) and YEALRVANEVTLN (SEQ ID NO: 3); or
d) a laminin fragment comprising or consisting of an amino acid sequence selected from the YIGSR (SEQ ID NO: 19), IKVAV (SEQ ID NO: 20), CCRRIKVAVWLC (SEQ ID NO: 21) and RGD.

5. The method of any preceding claim, wherein the hydrogel has a total gel density of from about 0.25 to about 0.50 g/cm³.

6. The method of any preceding claim, wherein the hydrogel has a gel hydration of from about 70% to about 90%.

7. The method of any preceding claim, wherein the hydrogel has a gel stiffness of from about 0.5 to about 35×10⁶ Pa.

8. The method of any preceding claim, wherein the hydrogel has a thickness of from about 0.1 to 12.0 mm.

9. The method of any preceding claim, wherein the hydrogel has a collagen density of from about 0.07 to about 0.3 g/cm³.

10. The method of any preceding claim, wherein the hydrogel is in the form of a thin layer or disc.

11. The method of any preceding claim, wherein the cell culture conditions comprise retaining the hydrogel on the curved surface in cell culture medium.

12. The method of claim 11, wherein the cell culture medium is Dulbecco's Modified Eagle Medium (DMEM), Ham's F12 or DMEM-F12.

13. The method of claim 11 or 12, wherein the cell culture medium is serum free.

14. The method of any of claims 11 to 13, wherein the cell culture medium comprises retinoic acid.

15. The method of any of claims 11 to 14, wherein the cell culture medium comprises KTTKS lipopeptides in solution.

16. The method of any preceding claim, wherein the curved surface is a polystyrene, polyethylene, polyethylene terephthalate, polylactic acid, polycarbonate, acrylonitrile butadiene styrene, agarose, a hydrogel or a glass surface, optionally wherein the glass surface is a borosilicate glass surface.

17. The method of any preceding claim, wherein the hydrogel is maintained on the curved surface under appropriate cell culture conditions for at least four days.

18. The method of any preceding claim, wherein the stromal cells are corneal stromal cells.

19. The method of claim 18, wherein the corneal stromal cells are corneal fibroblasts.

20. The method of any preceding claim, wherein the stromal cells are human.

21. The method of any of claims 2 to 20, wherein the stromal cells are from a first species and the extracellular matrix protein sequence is from a second species.

22. The method of any preceding claim, wherein the hydrogel comprising cell adhesion motifs is generated by extruding the hydrogel onto the curved surface, optionally wherein the hydrogel is generated using 3D bio-printing.

23. The method of any preceding claim, wherein the stromal cell tissue is subsequently decellularized.

24. A stromal cell tissue obtainable or obtained by a method according to claim 21 or 22.

25. A decellularized tissue obtainable or obtained by a method according to claim 23.

26. A stromal cell tissue having a minimum thickness of at least 50 μm, a minimum visible light transmittance value of at least 0.5 and a curvature of from about 0.04 to about 0.5 mm⁻¹, wherein the tissue comprises:
a) stromal cells from a first species and
b) trace amounts of:
(i) an extracellular matrix protein sequence from a second species, or a fragment or a variant thereof; or
(ii) a synthetic extracellular matrix protein sequence or a fragment or a variant thereof.

27. The stromal cell tissue of claim 26, wherein the first species is human and the second species is non-human.

28. The stromal cell tissue of claim 26 or 27, wherein the stromal cells are corneal stromal cells.

29. The stromal cell tissue of claim 28, wherein the corneal stromal cells are corneal fibroblasts.

30. A decellularized tissue having a minimum thickness of at least 50 μm, a minimum visible light transmittance value of at least 0.5 and a curvature of from about 0.04 and about 0.5 mm⁻¹, wherein the tissue comprises:
a) extracellular matrix protein sequences from a first species and
b) trace amounts of:
(i) an extracellular matrix protein sequence from a second species, or a fragment or a variant thereof; or
(ii) a synthetic extracellular matrix protein sequence or a fragment or a variant thereof.

31. The decellularized tissue of claim 30, wherein the first species is human, and the second species is non-human.

32. The tissue of any of claims 26 to 31, wherein the tissue has a thickness of from about 0.1 to 12.0 mm.

33. The tissue of any of claims 26 to 32, wherein the extracellular matrix protein sequence from a second species or synthetic extracellular matrix protein is collagen.

34. A tissue according to any of claims 24 to 33 for use in therapy.

35. A tissue according to any of claims 24 to 33 for use in keratoplasty.
